(19) United States
(12) Patent Application Publication
(10) Pub. No.: US 2021/0024900 A1
(43) Pub. Date: Jan. 28, 2021

(54) METHODS AND COMPOSITIONS RELATED TO INCREASED ROTAVIRUS PRODUCTION

(71) Applicants: UNIVERSITY OF GEORGIA RESEARCH FOUNDATION, INC., Athens, GA (US); MURDOCH CHILDREN'S RESEARCH INSTITUTE, Parkville, Victoria (AU)

(72) Inventors: Ralph A. TRIPP, Watkinsville, GA (US); Stephen M. TOMPKINS, Watkinsville, GA (US); Carl KIRKWOOD, Parkville, Victoria (AU)

(21) Appl. No.: 16/761,009

(22) PCT Filed: Nov. 2, 2018
(86) PCT No.: PCT/US2018/058849

§ 371 Date: May 1, 2020

Related U.S. Application Data
(60) Provisional application No. 62/581,020, filed on Nov. 2, 2017.

(51) Int. Cl. C12N 7/00; C12N 15/113

(52) U.S. Cl. CPC C12N 7/00; C12N 15/1137; C12N 2310/11; C12N 2310/531; C12Y 203/01; C12N 2310/141; C12N 2720/12351

(57) ABSTRACT

Disclosed are compositions and methods for increasing Rotavirus production.

This application claims the benefit of U.S. Provisional Application No. 62/581,020, filed on Nov. 2, 2017 which is incorporated herein by reference in its entirety.

### I. BACKGROUND

Vaccines are one of the most important defenses in the fight against infectious disease. The greater numbers of these vaccines are produced in cell culture. To achieve this, well characterized cell lines (e.g., Vero Cells) are (for example) grown in defined media formulations and then infected with live or live-attenuated viruses. Subsequently, the supernatant containing progeny of the original viral particles is collected and processed to create highly immunogenic doses of vaccine that can then be distributed amongst the population.

Currently, a complex set of factors (population dynamics, bioproduction, costs, etc.) limit the ability to provide adequate immunization coverage worldwide. In particular, bioproduction of vaccines can be expensive and the time required to provide needed quantities of a vaccine can significantly impact the medical benefit to society. This problem is particularly relevant for Rotavirus vaccines. Thus, new technologies are needed that increase vaccine production at greatly reduced costs.

### II. SUMMARY

1. Disclosed are methods and compositions related to increasing Rotavirus production in cells. The disclosed methods and compositions comprise reducing the expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPP5C, MET, SELM, TSPYL2, TSARG6, NDUFB2, PLAU, ADORA2B, FLJ22875, HMMR, NRK, LRIT3, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, DKFZP434K046, C9ORF112, and/or PIR51 genes the reduction of which increases Rotaviral production.

2. In one aspect, disclosed herein are cells comprising reduced expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPP5C, MET, SELM, TSPYL2, TSARG6, NDUFB2, PLAU, ADORA2B, FLJ22875, HMMR, NRK, LRIT3, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, DKFZP434K046, C9ORF112, and/or PIR51.

3. In one aspect, disclosed herein are methods of increasing Rotavirus production of one or more Rotaviruses comprising infecting a cell with a Rotavirus; wherein the cell comprises reduced expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPPSC, MET, SELM, TSPYL2, TSARG6, NDUFB2, PLAU, ADORA2B, FLJ22875, HMMR, NRK, LRIT3, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, DKFZP434K046, C9ORF112, and/or PIR51 genes.

### III. BRIEF DESCRIPTION OF THE DRAWINGS

4. The accompanying drawings, which are incorporated in and constitute a part of this specification, illustrate several embodiments and together with the description illustrate the disclosed compositions and methods.

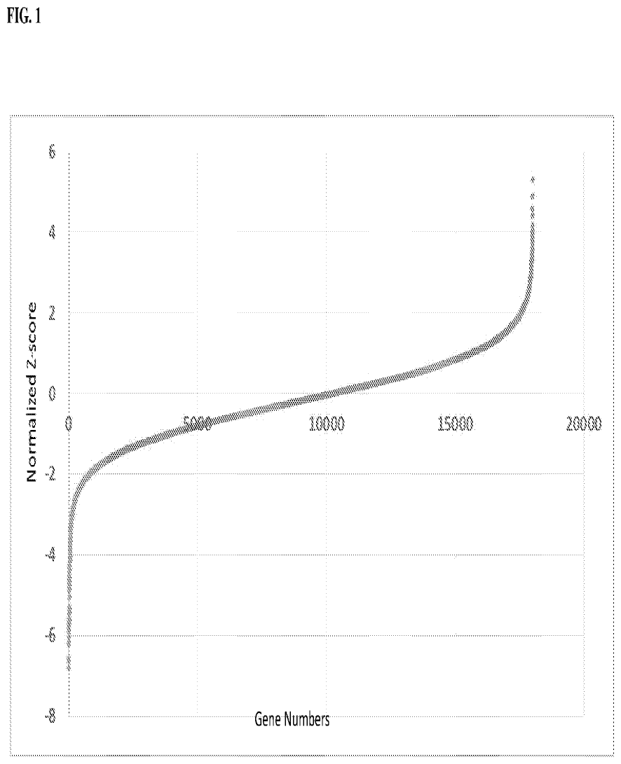

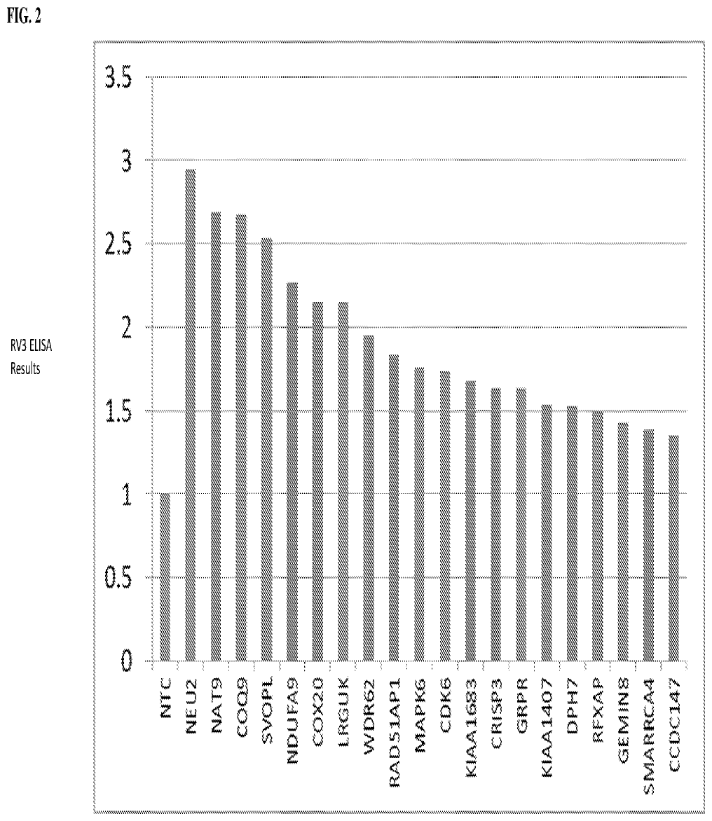

5. FIG. 1 shows the Z scores for the genome-wide RNAi screen designed to detect host gene modulation events that i) enhance, or ii) inhibit rotavirus replication in MA104 cells. Seventy-six gene suppression events significantly enhanced rotavirus replication (Z score greater than or equal to 3.0) as judged by ELISA. One hundred and twenty-one gene suppression events significantly decrease RV3 production.

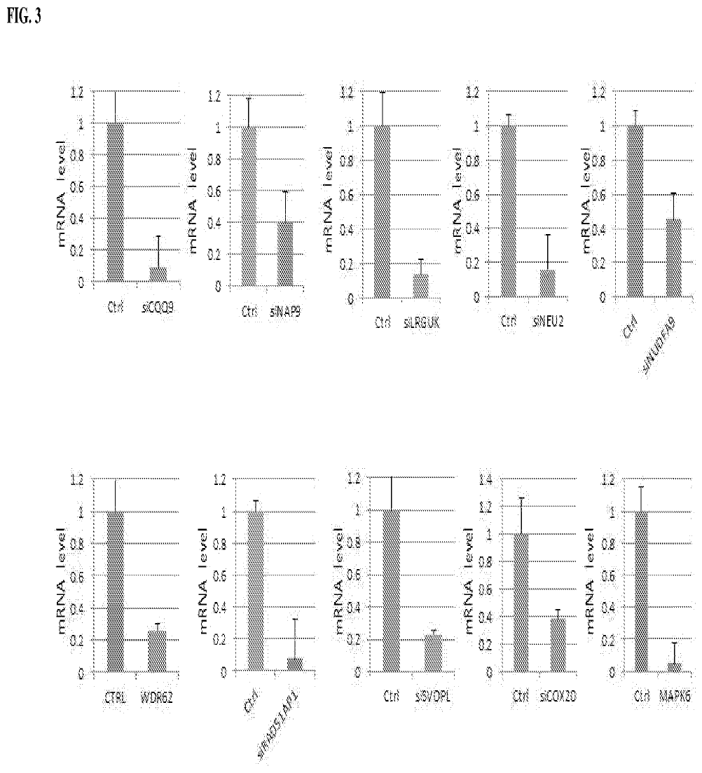

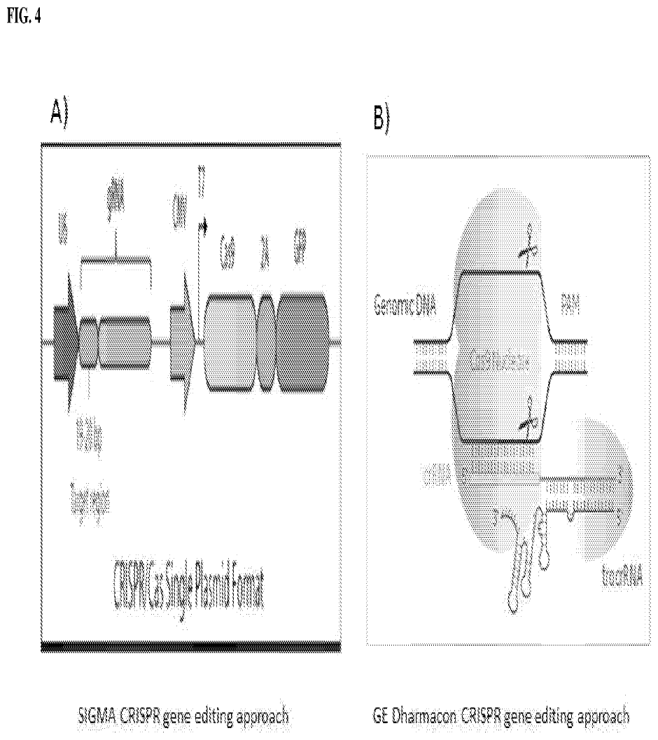

6. FIG. 2 shows how suppression of the top twenty gene targets affects rotavirus production in Vero cells. Y axis represents O.D. readings from ELISA readout. X axis identifies gene targeted by RNAi. “NTC”=non-targeting control.

7. FIG. 3 shows the effects of siRNA transfection on target gene levels in cells. Y axis represents the measured mRNA level. X axis identifies the control and target gene signals.

8. FIG. 4 shows two different approaches of CRISPR gene editing. The Sigma CRISPR system co-expresses gRNA and Cas9, together with a GFP marker protein for cell sorting. B) The GE Dharmacon system co-transfection of gRNA (cRNA:tracRNA) and a Cas9 plasmid.

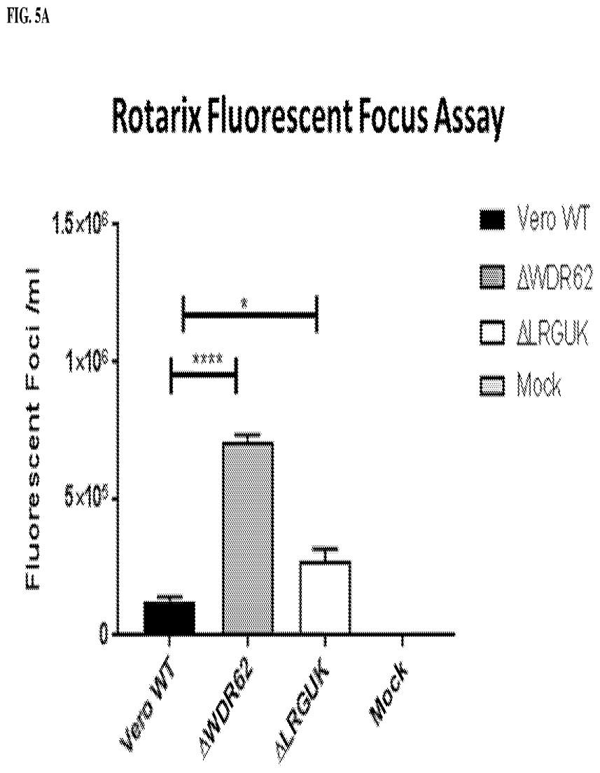

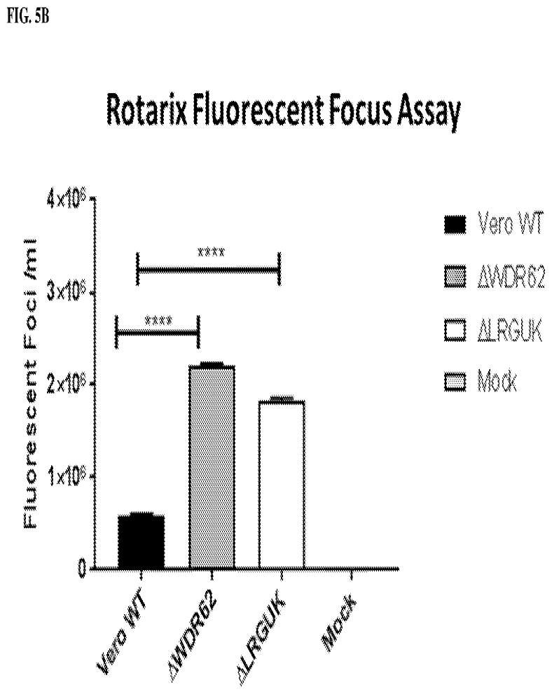

9. FIGS. 5A and 5B show that WT/KO Vero cells were infected with Rotarix (MOI 0.2) in 96-well format for 3 days (5A) or 5 days (5B) followed by transfer of supernatant to fresh cells (WT/KO) for 16 h. Cells were fixed with 4% formalin and then stained for RV antigen using a anti-RV rabbit polyclonal serum. Cells (n>20,000) were imaged on Arrayscan VTI. Data represent±SEM from six independent replicates. Differences in fluorescent foci were compared using one-way ANOVA *p<0.01; ****p<0.0001.

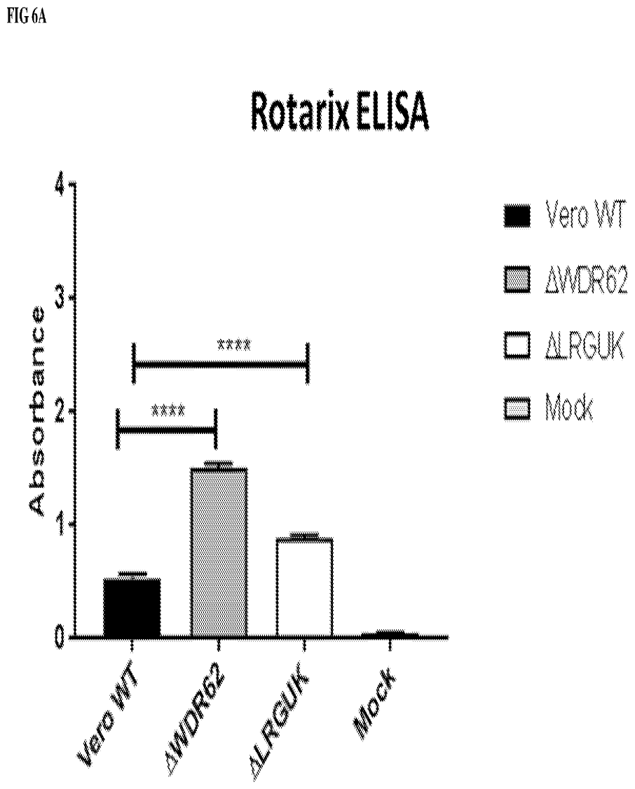

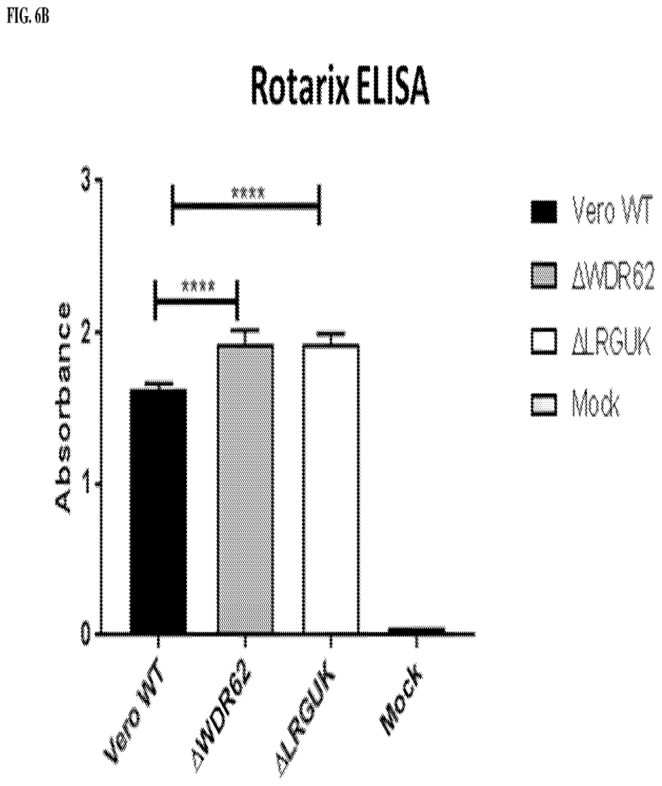

10. FIGS. 6A and 6B show that WT/KO Vero cells were infected with Rotarix (MOI 0.1) in 96-well format for 3 days (6A) or 5 days (6B) followed by transfer of supernatant to fresh cells for 16 h. Supernatants were collected for ELISA of RV antigen using a anti-RV rabbit polyclonal serum. Data represent±SEM from six independent replicates. Differences in absorbance were compared using one-way ANOVA ****p<0.0001.

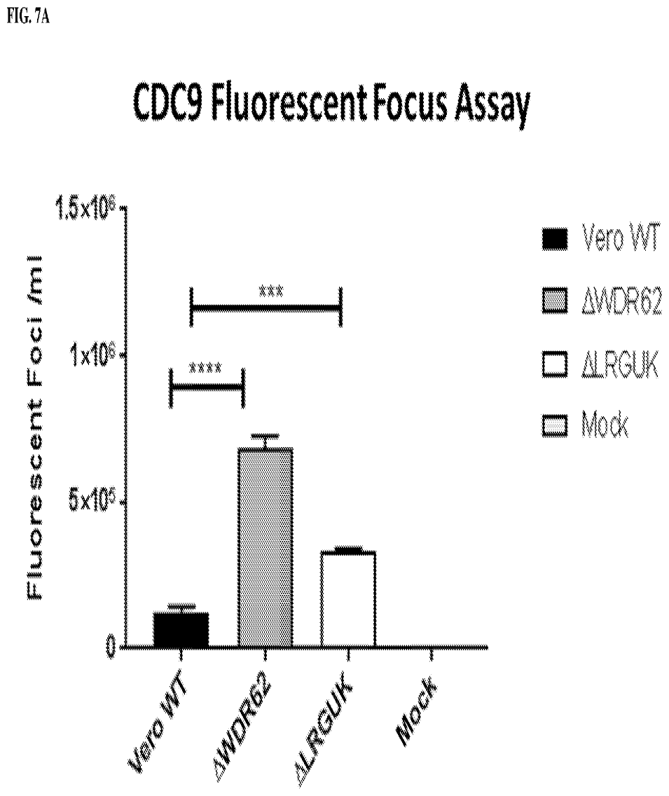

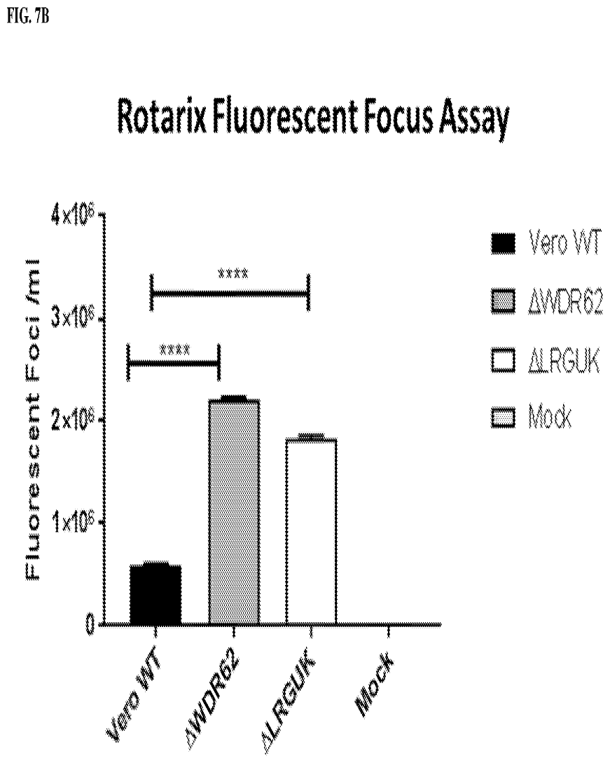

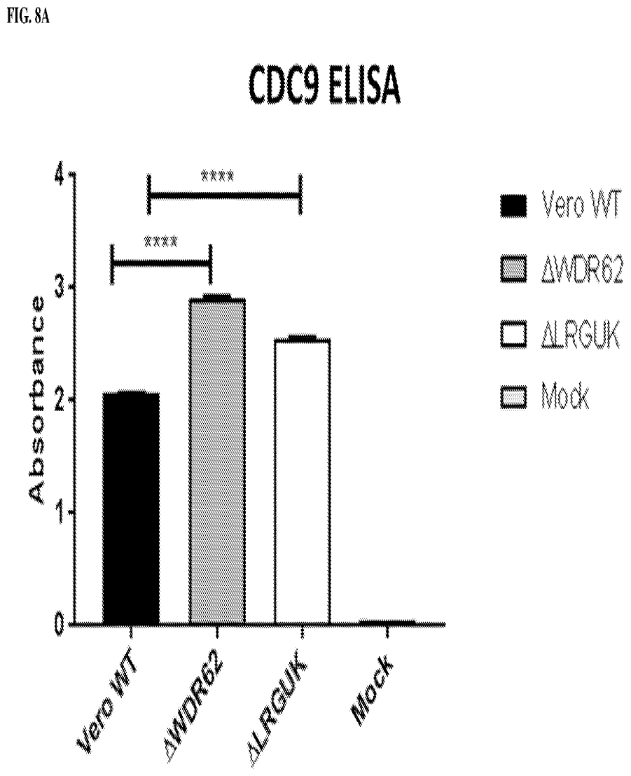

11. FIG. 7A and 7B show that WT/KO Vero cells were infected with CDC9 (MOI 0.1) in 96-well format for 3 days (7A) or 5 days (7B) followed by transfer of supernatant to fresh cells for 16 h. Cells were fixed with 4% formalin and then stained for RV antigen using a anti-RV rabbit polyclonal serum. Cells (n>20,000) were imaged on Arrayscan VTI. Data represent±SEM from six independent replicates. Differences in fluorescent foci were compared using one-way ANOVA ***p<0.01, ****p<0.0001.

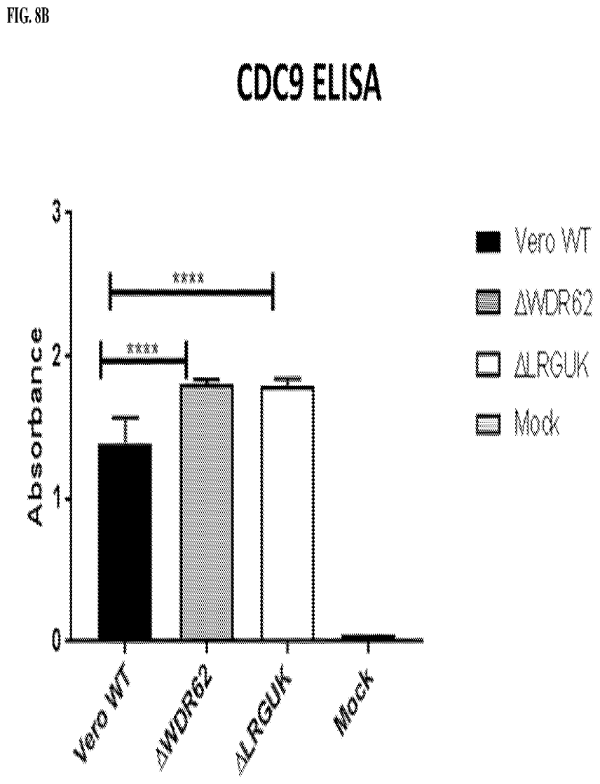

12. FIGS. 8A and 8B show that WT/KO Vero cells were infected with CDC9 (MOI 0.1) in 96-well format for 3 days (8A) or 5 days (8B) followed by transfer of supernatant to fresh cells for 16 h. Supernatants were collected for ELISA of RV antigen using a anti-RV rabbit polyclonal serum. Data represent±SEM from six independent replicates. Differences in absorbance were compared using one-way ANOVA ****p<0.0001.

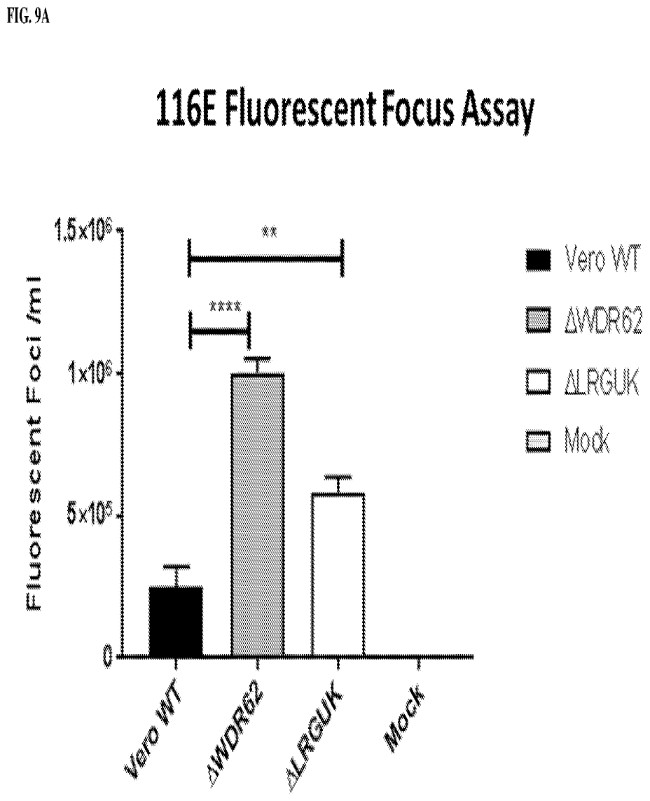

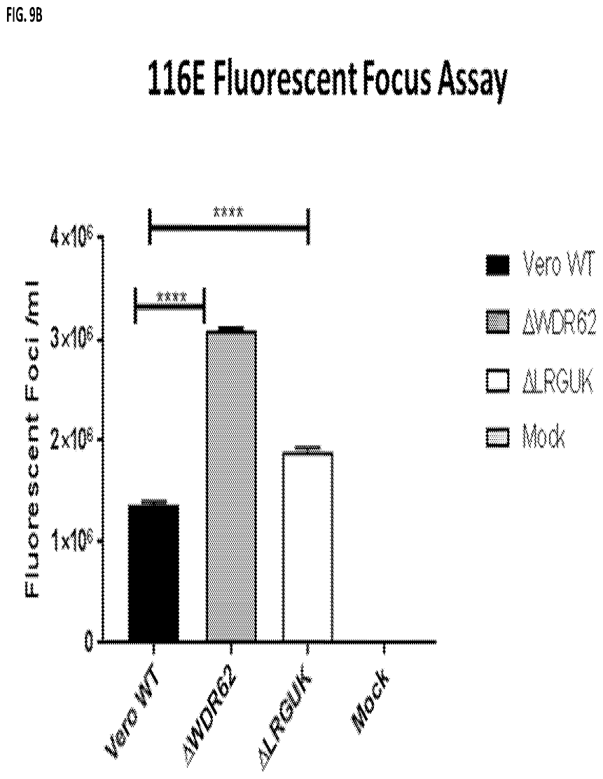

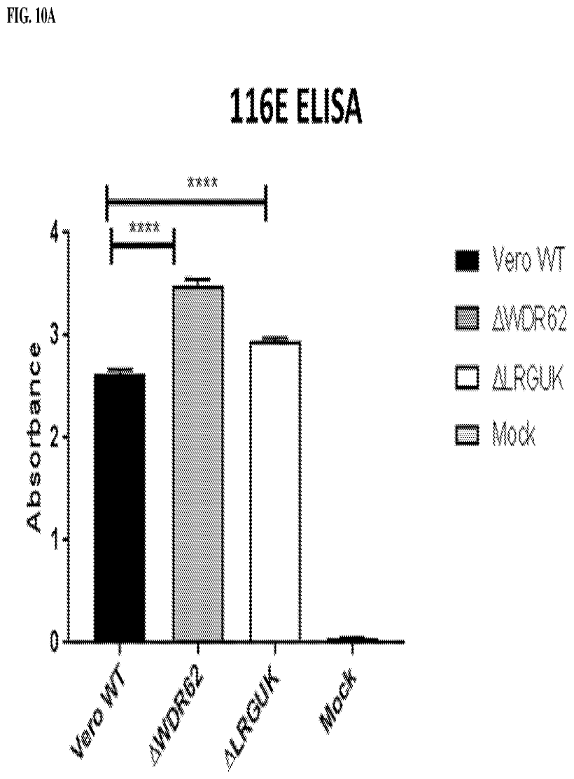

13. FIGS. 9A and 9B show that WT/KO Vero cells were infected with 116E (MOI 0.1) in 96-well format for 3 days (9A) or 5 days (9B) followed by transfer of supernatant to fresh cells for 16 h. Cells were fixed with 4% formalin and then stained for RV antigen using a anti-RV rabbit polyclonal serum. Cells (n>20,000) were imaged on Arrayscan VTI. Data represent±SEM from six independent replicates. Differences in fluorescent foci were compared using one-way ANOVA **p<0.01, ****p<0.0001.

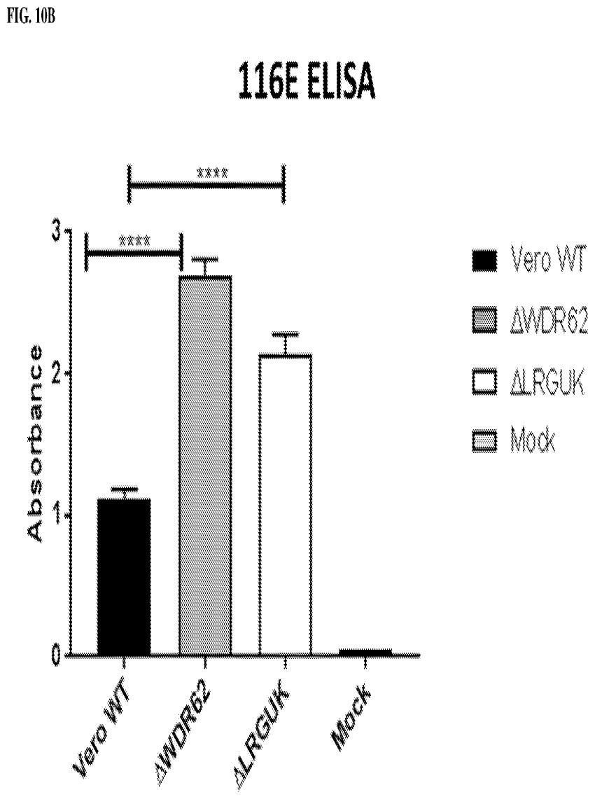

14. FIGS. 10A and 10B show WT/KO Vero cells were infected with 116E (MOI 0.1) in 96-well format for 3 days (10A) or 5 days (10B) followed by transfer of supernatant to fresh cells for 16 h. Supernatants were collected for ELISA of RV antigen using a anti-RV rabbit polyclonal serum. Data represent±SEM from six independent replicates. Differences in absorbance were compared using one-way ANOVA, ****p<0.0001

### IV. DETAILED DESCRIPTION

15. Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

16. As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

17. Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

18. In this specification and in the claims which follow, reference will be made to a number of terms which shall be defined to have the following meanings:

19. “Optional” or “optionally” means that the subsequently described event or circumstance may or may not occur, and that the description includes instances where said event or circumstance occurs and instances where it does not.

20. The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

21. In the context of this document, the term “target” or “target gene” or “hit” refers to any gene, including protein-encoding genes and non-coding RNAs (e.g., a miRNA) that (when modulated) positively or negatively alters some aspect of virus or biomolecule production. Target genes include endogenous host genes, pathogen (e.g., viral) genes, and transgenes.

22. The term “modulates” or “modulation” refers to the alteration of the regulation, expression or activity of a gene. In general, it is understood by those in the field that the term “modulation” includes increasing the expression or activity of a gene, decreasing the expression or activity of a gene, as well as altering the specificity or function of a gene. Modulating the expression or activity of a gene can be achieved by a number of means including altering one or more of the following: 1) gene copy number, 2) transcription or translation of a gene, 3) the transcript stability or longevity, 4) the number of copies of an mRNA or miRNA, 5) the availability of a non-coding RNA or non-coding RNA target site, 6) the position or degree of post-translational modifications on a protein, 7) the activity of a protein, and other mechanisms. Modulation can result in a significant reduction in target gene activity (e.g., at least 5%, at least 10%, at least 20% or greater reduction) or an increase in target gene activity (e.g., at least 10%, at least 20%, or greater increase). Furthermore, it is understood by those in the field that modulation of one or more genes can subsequently lead to the modulation of multiple genes (e.g., miRNAs).

23. Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Methods of Increasing Rotavirus Production

24. Rotavirus vaccines are used to protect human health and ensure food security. Unfortunately, current manufacturing capabilities are limited and costly, thereby placing significant portions of the human and agricultural animal populations at risk. To address this problem, what are needed are methods of increasing Rotaviral titers to enhance viral vaccine production. Accordingly, in one aspect, disclosed herein are methods of increasing Rotavirus production of one or more Rotaviruses and/or virus strains.

25. In the context of this document the term “vaccine” refers to an agent, including but not limited to a peptide or modified peptide, a protein or modified protein, a live virus, a live attenuated virus, an inactivated or killed virus, a virus-like particle (VLP), or any combination thereof, that is used to stimulate the immune system of an animal or human in order to provide protection against e.g., an infectious agent. Vaccines frequently act by stimulating the production of an antibody, an antibody-like molecule, or a cellular immune response in the subject(s) that receive such treatments.

26. The term “virus production” can refer to production of a live virus, or an attenuated virus, and/or a VLP. Production can occur by a multitude of methods including 1) production in an organism (e.g., an egg), a cultured cell (e.g., Vero cells), or in vitro (e.g., via a cell lysate).

27. Vaccines can be generated by a variety of means. In one instance, cells from any number of sources including but not limited to human, non-human primate, canine, and avian are first cultured in an appropriate environment (e.g., a cell or tissue culture plate or flask) to a desired density. Subsequently, viral seed stocks (e.g., rotavirus) are added to the culture where they infect cells. Infected cells are then transferred to a bioreactor (e.g., a single use bioreactor) where the virus replicates and expands in number. After a suitable period of time, the cells and cell particulate are separated from newly released viral particles and additional steps (e.g., purification, deactivation, concentration) are performed to further prepare the material for use as a vaccine.

28. With regard to the growth of the virus, the host cell makes a critical contribution to viral replication, contributing functions related to viral entry, genome replication, avoidance of the host immune system, and more.

29. Accordingly, and in one aspect, disclosed herein are methods of increasing Rotavirus production disclosed herein comprise infecting a cell with a Rotavirus; wherein the infected cell comprises reduced expression of at least one or more genes from Table 1 whose expression represses Rotaviral production. In other words, disclosed herein are methods of increasing Rotavirus production comprise infecting a cell with a Rotavirus; wherein the infected cell comprises genes that when modulated (individually or in combinations) enhance the production of rotavirus or rotavirus antigen in a cell or cell line (Table I). For example, disclosed herein, expression of ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPP5C, MET, SELM, TSPYL2, TSARG6, NDUFB2, PLAU, ADORA2B, FLJ22875, HMMR, NRK, LRIT3, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, DKFZP434K046, C9ORF112, and/or PIR51 negatively impact Rotaviral production. Accordingly, disclosed herein are methods of increasing Rotavirus production disclosed herein comprise infecting a cell with a Rotavirus; wherein the infected cell comprises reduced expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPP5C, MET, SELM, TSPYL2, TSARG6, NDUFB2, PLAU, ADORA2B, FLJ22875, HMMR, NRK, LRIT3, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, DKFZP434K046, C9ORF112, and/or PIR51 genes.

30. As disclosed herein, the disclosed methods can comprise the reduced expression of any combination of one, two, three, four, five, six, seven, eight, nine, 10, 11, 12, 13, 14, 15, 16, 17, 18, 19, 20, 21, 22, 23, 24, 25, 30, 35, 40, 45, 50, 55, 60, 65, 70, 71, 72, 73, 74, 75, or all 76 of the disclosed genes (i.e., ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51). For example, the cell can comprise reduced expression of NAT9 alone or in combination with any one, two, three, four, five, six, seven, eight, nine, 10, 11, 12, 13, 14, 15, 16, 17, 18, 19, 20, 21, 22, 23, 24, 25, 30, 35, 40, 45, 50, 55, 60, 65, 70, 71, 72, 73, 74, 75, 76, or 77 other of the selected genes. Thus, for example, in one aspect, disclosed herein are method of increasing Rotavirus production comprising infecting a cell with Rotavirus; wherein the cell comprises reduced expression of ZNF205; NEU2; NAT9; SVOPL; COQ9, BTN2A1, PYCR1, EP300, SEC61G; NDUFA9; RAD51AP1; COX20; MAPK6; WDR62; LRGUK; CDK6; KIAA1683; CRISP3; GRPR; DPH7; GEMIN8; KIAA1407; RFXAP; SMARRCA4; CCDC147; AACS; CDK9; C7ORF26; ZDHHC14; RNUT1; GAB1; EMC3; FAM96A; FAM36A; LOC55831; LOC136306; DEFB126; MGC955; EPHX2; SRGAP1; PPPSC; MET; SELM; TSPYL2; TSARG6; NDUFB2; PLAU; FLJ36888; ADORA2B; FLJ22875; HMMR; NRK, LRIT3; FLJ44691; GPR154; ZGPAT; DRD1; FLJ27505; EDG5; SNRNP40; HPRP8BP; GPA33; JDP2; FLJ20010; FOXJ1; SCT; CHD1L; SULT1C1; STN2; MRS2L; RAD51AP1; DPH7; CLPP; ZNF37; AP3B2; DEGS2; PIR; D2LIC; CNTF; PAM; MYH9; PRPF4; SLC4A11; LRRCC1; FZD9; GPR43; LTF; ARIH1; PIK3R3; PTGFRN; KIAA1764; C19ORF14; FLNA; FLJ32786; DKFZP434K046; C9ORF112; PIR51; NAT9 and NEU2; NAT9 and SVOPL; NAT9 and COQ9; NAT9 and NDUFA9; NAT9 and RAD51AP1; NAT9 and COX20; NAT9 and MAPK6; NAT9 and WDR62; NAT9 and LRGUK; NAT9 and CDK6; NAT9 and KIAA1683; NAT9 and CRISP3; NAT9 and GRPR; NAT9 and DPH7; NAT9 and GEMIN8; NAT9 and KIAA1407; NAT9 and RFXAP; NAT9 and SMARRCA4; NAT9 and CCDC147; NAT9 and AACS; NAT9 and CDK9; NAT9 and C7ORF26; NAT9 and ZDHHC14; NAT9 and RNUT1; NAT9 and GAB1; NAT9 and EMC3; NAT9 and FAM96A; NAT9 and FAM36A; NAT9 and LOC55831; NAT9 and LOC136306; NAT9 and DEFB126; NAT9 and MGC955; NAT9 and EPHX2; NAT9 and SRGAP1; NAT9 and PPP5C; NAT9 and MET; NAT9 and SELM; NAT9 and TSPYL2; NAT9 and TSARG6; NAT9 and NDUFB2; NAT9 and PLAU; NAT9 and FLJ36888; NAT9 and ADORA2B; NAT9 and FLJ22875; NAT9 and HMMR; NAT9 and NRK; NAT9 and FLJ44691; NAT9 and GPR154; NAT9 and ZGPAT; NAT9 and DRD1; NAT9 and FLJ27505; NAT9 and EDG5; NAT9 and SNRNP40; NAT9 and HPRP8BP; NAT9 and GPA33; NAT9 and JDP2; NAT9 and FLJ20010; NAT9 and FOXE; NAT9 and SCT; NAT9 and CHD1L; NAT9 and SULT1C1; NAT9 and STN2; NAT9 and MRS2L; NAT9 and RAD51AP1; NAT9 and DPH7; NAT9 and CLPP; NAT9 and ZNF37; NAT9 and AP3B2; NAT9 and COQ9; NAT9 and DEGS2; NAT9 and PIR; NAT9 and D2LIC; NAT9 and CNTF; NAT9 and PAM; NAT9 and MYH9; NAT9 and PRPF4; NAT9 and SLC4A11; NAT9 and LRRCC1; NAT9 and FZD9; NAT9 and GPR43; NAT9 and LTF; NAT9 and ARIH1; NAT9 and PIK3R3; NAT9 and PTGFRN; NAT9 and KIAA1764; NAT9 and C19ORF14; NAT9 and FLNA; NAT9 and FLJ32786; NAT9 and DKFZP434K046; NAT9 and C9ORF112; NAT9 and PIR51; NEU2 and SVOPL; NEU2 and COQ9; NEU2 and NDUFA9; NEU2 and RAD51AP1; NEU2 and COX20; NEU2 and MAPK6; NEU2 and WDR62; NEU2 and LRGUK; NEU2 and CDK6; NEU2 and KIAA1683; NEU2 and CRISP3; NEU2 and GRPR; NEU2 and DPH7; NEU2 and GEMIN8; NEU2 and KIAA1407; NEU2 and RFXAP; NEU2 and SMARRCA4; NEU2 and CCDC147 SVOPL and COQ9; SVOPL and NDUFA9; SVOPL and RAD51AP1; SVOPL and COX20; SVOPL and MAPK6; SVOPL and WDR62; SVOPL and LRGUK; SVOPL and CDK6; SVOPL and KIAA1683; SVOPL and CRISP3; SVOPL and GRPR; SVOPL and DPH7; SVOPL and GEMIN8; SVOPL and KIAA1407; SVOPL and RFXAP; SVOPL and SMARRCA4; SVOPL and CCDC147; COQ9 and NDUFA9; COQ9 and RAD51AP1; COQ9 and COX20; COQ9 and MAPK6; COQ9 and WDR62; COQ9 and LRGUK; COQ9 and CDK6; COQ9 and KIAA1683; COQ9 and CRISP3; COQ9 and GRPR; COQ9 and DPH7; COQ9 and GEMIN8; COQ9 and KIAA1407; COQ9 and RFXAP; COQ9 and SMARRCA4; COQ9 and CCDC147 ; NDUFA9 and RAD51AP1; NDUFA9 and COX20; NDUFA9 and MAPK6; NDUFA9 and WDR62; NDUFA9 and LRGUK; NDUFA9 and CDK6; NDUFA9 and KIAA1683; NDUFA9 and CRISP3; NDUFA9 and GRPR; NDUFA9 and DPH7; NDUFA9 and GEMIN8; NDUFA9 and KIAA1407; NDUFA9 and RFXAP; NDUFA9 and SMARRCA4; NDUFA9 and CCDC147 ; RAD51AP1 and COX20; RAD51AP1 and MAPK6; RAD51AP1 and WDR62; RAD51AP1 and LRGUK; RAD51AP1 and CDK6; RAD51AP1 and KIAA1683; RAD51AP1 and CRISP3; RAD51AP1 and GRPR; RAD51AP1 and DPH7; RAD51AP1 and GEMIN8; RAD51AP1 and KIAA1407; RAD51AP1 and RFXAP; RAD51AP1 and SMARRCA4; RAD51AP1 and CCDC147; COX20 and MAPK6; COX20 and WDR62; COX20 and LRGUK; COX20 and CDK6; COX20 and KIAA1683; COX20 and CRISP3; COX20 and GRPR; COX20 and DPH7; COX20 and GEMIN8; COX20 and KIAA1407; COX20 and RFXAP; COX20 and SMARRCA4; COX20 and CCDC147; MAPK6 and WDR62; MAPK6 and LRGUK; MAPK6 and CDK6; MAPK6 and KIAA1683; MAPK6 and CRISP3; MAPK6 and GRPR; MAPK6 and DPH7; MAPK6 and GEMIN8; MAPK6 and KIAA1407; MAPK6 and RFXAP; MAPK6 and SMARRCA4; MAPK6 and CCDC147; WDR62 and LRGUK; WDR62 and CDK6; WDR62 and KIAA1683; WDR62 and CRISP3; WDR62 and GRPR; WDR62 and DPH7; WDR62 and GEMIN8; WDR62 and KIAA1407; WDR62 and RFXAP; WDR62 and SMARRCA4; WDR62 and CCDC147; LRGUK and CDK6; LRGUK and KIAA1683; LRGUK and CRISP3; LRGUK and GRPR; LRGUK and DPH7; LRGUK and GEMIN8; LRGUK and KIAA1407; LRGUK and RFXAP; LRGUK and SMARRCA4; LRGUK and CCDC147; CDK6 and KIAA1683; CDK6 and CRISP3; CDK6 and GRPR; CDK6 and DPH7; CDK6 and GEMIN8; CDK6 and KIAA1407; CDK6 and RFXAP; CDK6 and SMARRCA4; CDK6 and CCDC147; KIAA1683 and CRISP3; KIAA1683 and GRPR; KIAA1683 and DPH7; KIAA1683 and GEMIN8; KIAA1683 and KIAA1407; KIAA1683 and RFXAP; KIAA1683 and SMARRCA4; KIAA1683 and CCDC147; CRISP3 and GRPR; CRISP3 and DPH7; CRISP3 and GEMIN8; CRISP3 and KIAA1407; CRISP3 and RFXAP; CRISP3 and SMARRCA4; CRISP3 and CCDC147; GRPR and DPH7; GRPR and GEMIN8; GRPR and KIAA1407; GRPR and RFXAP; GRPR and SMARRCA4; GRPR and CCDC147; DPH7 and GEMIN8; DPH7 and KIAA1407; DPH7 and RFXAP; DPH7 and SMARRCA4; DPH7 and CCDC147; GEMIN8 and KIAA1407; GEMIN8 and RFXAP; GEMIN8 and SMARRCA4; GEMIN8 and CCDC147; KIAA1407 and RFXAP; KIAA1407 and SMARRCA4; KIAA1407 and CCDC147; RFXAP and SMARRCA4; RFXAP and CCDC147; SMARRCA4 and CCDC147; ZNF205 and NEU2; ZNF205 and ZNF205, NAT9; ZNF205 and SVOPL; ZNF205 and COQ9; ZNF205 and NDUFA9; ZNF205 and RAD51AP1; ZNF205 and COX20; ZNF205 and MAPK6; ZNF205 and WDR62; ZNF205 and LRGUK; ZNF205 and CDK6; ZNF205 and KIAA1683; ZNF205 and CRISP3; ZNF205 and GRPR; ZNF205 and DPH7; ZNF205 and GEMIN8; ZNF205 and KIAA1407; ZNF205 and RFXAP; ZNF205 and SMARRCA4; ZNF205 and CCDC147; ZNF205 and AACS; ZNF205 and CDK9; ZNF205 and C7ORF26; ZNF205 and ZDHHC14; ZNF205 and RNUT1; ZNF205 and GAB 1; ZNF205 and EMC3; ZNF205 and FAM96A; ZNF205 and FAM36A; ZNF205 and LOC55831; ZNF205 and LOC136306; ZNF205 and DEFB126; ZNF205 and MGC955; ZNF205 and EPHX2; ZNF205 and SRGAP1; ZNF205 and PPP5C; ZNF205 and MET; ZNF205 and SELM; ZNF205 and TSPYL2; ZNF205 and TSARG6; ZNF205 and NDUFB2; ZNF205 and PLAU; ZNF205 and FLJ36888; ZNF205 and ADORA2B; ZNF205 and FLJ22875; ZNF205 and HMMR; ZNF205 and NRK; ZNF205 and FLJ44691; ZNF205 and GPR154; ZNF205 and ZGPAT; ZNF205 and DRD1; ZNF205 and FLJ27505; ZNF205 and EDG5; ZNF205 and SNRNP40; ZNF205 and HPRP8BP; ZNF205 and GPA33; ZNF205 and JDP2; ZNF205 and FLJ20010; ZNF205 and FOXJ1; ZNF205 and SCT; ZNF205 and CHD1L; ZNF205 and SULT1C1; ZNF205 and STN2; ZNF205 and MRS2L; ZNF205 and RAD51AP1; ZNF205 and DPH7; ZNF205 and CLPP; ZNF205 and ZNF37; ZNF205 and AP3B2; ZNF205 and COQ9; ZNF205 and DEGS2; ZNF205 and PIR; ZNF205 and D2LIC; ZNF205 and CNTF; PAM; ZNF205 and MYH9; ZNF205 and PRPF4; ZNF205 and SLC4A11; ZNF205 and LRRCC1; ZNF205 and FZD9; ZNF205 and GPR43; ZNF205 and LTF; ZNF205 and ARIH1; ZNF205 and PIK3R3; ZNF205 and PTGFRN; ZNF205 and KIAA1764; ZNF205 and C19ORF14; ZNF205 and FLNA; ZNF205 and FLJ32786; ZNF205 and DKFZP434K046; ZNF205 and C9ORF112; ZNF205 and PIR51; ZNF205, NAT9 and NEU2; ZNF205, NAT9 and SVOPL; ZNF205, NAT9 and COQ9; ZNF205, NAT9 and NDUFA9; ZNF205, NAT9 and RAD51AP1; ZNF205, NAT9 and COX20; ZNF205, NAT9 and MAPK6; ZNF205, NAT9 and WDR62; ZNF205, NAT9 and LRGUK; ZNF205, NAT9 and CDK6; ZNF205, NAT9 and KIAA1683; ZNF205, NAT9 and CRISP3; ZNF205, NAT9 and GRPR; ZNF205, NAT9 and DPH7; ZNF205, NAT9 and GEMIN8; ZNF205, NAT9 and KIAA1407; ZNF205, NAT9 and RFXAP; ZNF205, NAT9 and SMARRCA4; ZNF205, NAT9 and CCDC147; ZNF205, NAT9 and AACS; ZNF205, NAT9 and CDK9; ZNF205, NAT9 and C7ORF26; ZNF205, NAT9 and ZDHHC14; ZNF205, NAT9 and RNUT1; ZNF205, NAT9 and GAB1; ZNF205, NAT9 and EMC3; ZNF205, NAT9 and FAM96A; ZNF205, NAT9 and FAM36A; ZNF205, NAT9 and LOC55831; ZNF205, NAT9 and LOC136306; ZNF205, NAT9 and DEFB126; ZNF205, NAT9 and MGC955; ZNF205, NAT9 and EPHX2; ZNF205, NAT9 and SRGAP1; ZNF205, NAT9 and PPP5C; ZNF205, NAT9 and MET; ZNF205, NAT9 and SELM; ZNF205, NAT9 and TSPYL2; ZNF205, NAT9 and TSARG6; ZNF205, NAT9 and NDUFB2; ZNF205, NAT9 and PLAU; ZNF205, NAT9 and FLJ36888; ZNF205, NAT9 and ADORA2B; ZNF205, NAT9 and FLJ22875; ZNF205, NAT9 and HMMR; ZNF205, NAT9 and NRK; ZNF205, NAT9 and FLJ44691; ZNF205, NAT9 and GPR154; ZNF205, NAT9 and ZGPAT; ZNF205, NAT9 and DRD1; ZNF205, NAT9 and FLJ27505; ZNF205, NAT9 and EDG5; ZNF205, NAT9 and SNRNP40; ZNF205, NAT9 and HPRP8BP; ZNF205, NAT9 and GPA33; ZNF205, NAT9 and JDP2; ZNF205, NAT9 and FLJ20010; ZNF205, NAT9 and FOXJ1; ZNF205, NAT9 and SCT; ZNF205, NAT9 and CHD1L; ZNF205, NAT9 and SULT1C1; ZNF205, NAT9 and STN2; ZNF205, NAT9 and MRS2L; ZNF205, NAT9 and RAD51AP1; ZNF205, NAT9 and DPH7; ZNF205, NAT9 and CLPP; ZNF205, NAT9 and ZNF37; ZNF205, NAT9 and AP3B2; ZNF205, NAT9 and COQ9; ZNF205, NAT9 and DEGS2; ZNF205, NAT9 and PIR; ZNF205, NAT9 and D2LIC; ZNF205, NAT9 and CNTF; ZNF205, NAT9 and PAM; ZNF205, NAT9 and MYH9; ZNF205, NAT9 and PRPF4; ZNF205, NAT9 and SLC4A11; ZNF205, NAT9 and LRRCC1; ZNF205, NAT9 and FZD9; ZNF205, NAT9 and GPR43; ZNF205, NAT9 and LTF; ZNF205, NAT9 and ARIH1; ZNF205, NAT9 and PIK3R3; ZNF205, NAT9 and PTGFRN; ZNF205, NAT9 and KIAA1764; ZNF205, NAT9 and C19ORF14; ZNF205, NAT9 and FLNA; ZNF205, NAT9 and FLJ32786; ZNF205, NAT9 and DKFZP434K046; ZNF205, NAT9 and C9ORF112; and ZNF205, NAT9 and PIR51. Any other combination of two or more of the disclosed genes ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, DEFB126, MGC955, EPHX2, SRGAP1, PPP5C, MET, SELM, TSPYL2, TSARG6, NDUFB2, PLAU, ADORA2B, FLJ22875, HMMR, NRK, FLJ44691, GPR154, ZGPAT, DRD1, FLJ27505, EDG5, SNRNP40, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, KIAA1764, C19ORF14, DKFZP434K046, C9ORF112, and/or PIR51 is specifically disclosed herein.

31. As used herein, “increased viral production,” “increasing viral production,” “increased Rotaviral production,” and “increasing Rotaviral production,” refer to a change in viral titers resulting in more virus being produced.

32. The disclosed methods can be performed with any cell that can be infected with Rotavirus. In one aspect, the cells can be of mammalian origin (including, human, simian, porcine, bovine, equine, canine, feline, rodent (e.g., rabbit, rat, mouse, and guinea pig), and non-human primate) or avian including chicken, duck, ostrich, and turkey cells. It is further contemplated that the cell can be a cell of an established mammalian cell line including, but not limited to MA104 cells, VERO cells, Madin-Darby Canine Kidney (MDCK) cells, HEp-2 cells, HeLa cells, HEK293 cells, MRC-5 cells, WI-38 cells, EB66, and PER C6 cells.

33. Rotavirus is a virus comprising many species, serotypes, subtypes, strains, variants, and reassortants known in the art. The term “rotavirus” is intended to include any of the current or future rotavirus that can be used in vaccine production. These include any and all wild type strains, parental strains, or attenuated strains such as the strains that make up the current commercial vaccines, CDC9 strain, 116E strain, RotaTeq (G1P7, G2P7, G3P7, G4P7, G6P1A) and Rotarix (89-12/G11181) strains), the RV3-BB strain which is currently under development at BioFarma, Indonesia (see Danchin, M. et al (2013) “Phase I trial of RV3-BB rotavirus vaccine: A human neonatal rotavirus vaccine.” Vaccine 31:2610-2616), as well as the CDC-9 strain, a live attenuated human G1P RV strain which has recently gone through Phase 3 trials in India). Lastly, relevant strains include G9 variants. Additionally, in the context of this document, the term “rotavirus” includes any VLPs derived from any of the before mentioned strains or closely related viruses as well as current or future recombinant or engineered strains. Lastly, the term also includes any member of the family Reoviridae other than the known rotaviruses,

34. As noted above, it is understood and herein contemplated that the disclosed methods can work for any Rotavirus including all known Rotaviruses species (e.g., Rotavirus A, Rotavirus B, Rotavirus C, Rotavirus D, Rotavirus E, Rotavirus F, Rotavirus G, and Rotavirus H), viral strains, serotypes (P1, P2A, P2B, P2C, P3, P4, PSA, PSB, P6, P7, P8, P9, P10, P11, P12, P13, or P14), and variants including, but not limited to Rotavirual reassortants. It is further understood that the disclosed methods include superinfection (i.e., concurrent infection of multiple viral strains) of a single cell with one, two, three, four, five, six, seven, eight, nine, ten, or more species, strains, variants, reassortants, or serotypes of Rotavirus. Preferably, modulation of the gene(s) in the described list enhance the production of the RV3 vaccine strain of rotavirus. More preferably, modulation of the gene(s) in the described list enhance the production of G1P7, G2P7, G3P7, G4P7, G6P1A, 89-12/G11181, RV3-BB, CDC-9, and/or a G9 strain of rotavirus or rotavirus antigen in a cell or cell line that is used in rotavirus vaccine manufacturing.

35. The methods disclosed herein utilize a reduction in expression of a gene or its encoded protein to increase Rotaviral production. As used herein “reduced” or “decreased” expression refers to a change in the transcription of a gene, translation of an mRNA, or the activity of a protein encoded by a gene that results in less of the gene, translated mRNA, encoded protein, or protein activity relative to a control. Reduction in expression can be at least a 1, 2, 3, 4, 5, 6, 7, 8, 9, 10, 15, 20, 25, 30, 35, 40, 45, 50, 55, 60, 65, 70, 75, 80, 85, 86, 87, 88, 89, 90, 91, 92, 93, 94, 95, 96, 97, 98, 99, or 100% reduction of the gene expression, mRNA translation, protein expression, or protein activity relative to a control. For example, disclosed herein are methods of increasing Rotavirus production disclosed herein comprise infecting a cell with a Rotavirus; wherein the infected cell comprises at least 1, 2, 3, 4, 5, 6, 7, 8, 9, 10, 15, 20, 25, 30, 35, 40, 45, 50, 55, 60, 65, 70, 75, 80, 85, 86, 87, 88, 89, 90, 91, 92, 93, 94, 95, 96, 97, 98, 99, or 100% reduction of the expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, KIAA1764, C19ORF14, FLNA, FLJ32786, DKFZP434K046, C9ORF112, and/or PIR51 genes relative to a control.

36. It is further understood that one way of referring to a reduction rather than the percentage reduction is as a percentage of the control expression or activity. For example, a cell with at least a 15% reduction in the expression of a particular gene relative to a control would also be a gene with expression that is less than or equal to 85% of the expression of the control. Accordingly, in one aspect are methods wherein the gene expression, mRNA expression, protein expression, or protein activity is less than or equal to 95, 90, 89, 88, 87, 86, 85, 84, 83, 82, 81, 80, 75, 70, 65, 60, 55, 50, 45, 40, 35, 30, 25, 20, 15, 10, 9, 8, 7, 6, 5, 4, 3, 2, 1% of a control. Thus, disclosed herein are methods of increasing Rotavirus production disclosed herein comprise infecting a cell with a Rotavirus; wherein the infected cell comprises less than or equal to 95, 90, 89, 88, 87, 86, 85, 84, 83, 82, 81, 80, 75, 70, 65, 60, 55, 50, 45, 40, 35, 30, 25, 20, 15, 10, 9, 8, 7, 6, 5, 4, 3, 2, 1% reduction of the expression of at least one gene, mRNA, protein, or protein activity selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 relative to a control. For example, disclosed herein are methods of increasing Rotavirus production disclosed herein comprise infecting a cell with a Rotavirus; wherein the infected cell comprises less than or equal to 85% reduction of the expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 relative to a control.

37. It is understood and herein contemplated that the reduced expression can be achieved by any means known in the art including techniques that manipulate genomic DNA, messenger and/or non-coding RNA and/or proteins including but not limited to endogenous or exognenous control elements (e.g., small interfering RNAs (siRNA), small hairpin RNAs (shRNA), small molecule inhibitor, and antisense oligonucleotide) and/or mutations are present in or directly target the coding region of the gene, mRNA, or protein or are present in or target a regulator region operably linked to the gene, mRNA, or protein. As such, the technologies or mechanisms that can be employed to modulate a gene of interest include but are not limited to 1) technologies and reagents that target genomic DNA to result in an edited genome (e.g., homologous recombination to introduce a mutation such as a deletion into a gene, zinc finger nucleases, meganucleases, transcription activator-like effectors (e.g., TALENs), triplexes, mediators of epigenetic modification, and CRISPR and rAAV technologies), 2) technologies and reagents that target RNA (e.g. agents that act through the RNAi pathway, antisense technologies, ribozyme technologies), and 3) technologies that target proteins (e.g., small molecules, aptamers, peptides, auxin- or FKBP-mediated destabilizing domains, antibodies).

38. In one embodiment for targeting DNA, gene modulation is achieved using zinc finger nucleases (ZFNs). Synthetic ZFNs are composed of a custom designed zinc finger binding domain fused with e.g. a Fokl DNA cleavage domain. As these reagents can be designed/engineered for editing the genome of a cell, including, but not limited to, knock out or knock in gene expression, in a wide range of organisms, they are considered one of the standards for developing stable engineered cell lines with desired traits. Meganucleases, triplexes, CRISPR, and recombinant adeno-associated viruses have similarly been used for genome engineering in a wide array of cell types and are viable alternatives to ZFNs. The described reagents can be used to target promoters, protein-encoding regions (exons), introns, 5′ and 3′ UTRs, and more.

39. Another embodiment for modulating gene function utilizes the cell's endogenous or exogenous RNA interference (RNAi) pathways to target cellular messenger RNA. In this approach, gene targeting reagents include small interfering RNAs (siRNA) as well as microRNAs (miRNA). These reagents can incorporate a wide range of chemical modifications, levels of complementarity to the target transcript of interest, and designs (see U.S. Pat. No. 8,188,060) to enhance stability, cellular delivery, specificity, and functionality. In addition, such reagents can be designed to target diverse regions of a gene (including the 5′ UTR, the open reading frame, the 3′ UTR of the mRNA), or (in some cases) the promoter/enhancer regions of the genomic DNA encoding the gene of interest. Gene modulation (e.g., knockdown) can be achieved by introducing (into a cell) a single siRNA or miRNA or multiple siRNAs or miRNAs (i.e., pools) targeting different regions of the same mRNA transcript. Synthetic siRNA/miRNA delivery can be achieved by any number of methods including but not limited to 1) self-delivery (US Patent Application No 2009/0280567A1), 2) lipid-mediated delivery, 3) electroporation, or 4) vector/plasmid-based expression systems. An introduced RNA molecule may be referred to as an exogenous nucleotide sequence or polynucleotide.

40. Another gene targeting reagent that uses RNAi pathways includes exogenous small hairpin RNA, also referred to as shRNA. shRNAs delivered to cells via e.g., expression constructs (e.g., plasmids, lentiviruses) have the ability to provide long term gene knockdown in a constitutive or regulated manner, depending upon the type of promoter employed. In one preferred embodiment, the genome of a lentiviral particle is modified to include one or more shRNA expression cassettes that target a gene (or genes) of interest. Such lentiviruses can infect a cell intended for vaccine production, stably integrate their viral genome into the host genome, and express the shRNA(s) in a 1) constitutive, 2) regulated, or (in the case where multiple shRNA are being expressed) constitutive and regulated fashion. In this way, cell lines having enhanced Rotavirus production capabilities can be created. It is worth noting, that approaches that use siRNA or shRNA have the added benefit in that they can be designed to target individual variants of a single gene or multiple closely related gene family members. In this way, individual reagents can be used to modulate larger collections of targets having similar or redundant functions or sequence motifs. The skilled person will recognize that lentiviral constructs can also incorporate cloned DNA, or ORF expression constructs.

41. In another embodiment for modulating gene function, gene suppression can be achieved by large scale transfection of cells with miRNA mimics or miRNA inhibitors introduced into the cells.

42. In another embodiment, modulation takes place at the protein level. By example, knockdown of gene function at the protein level can be achieved by a number of means including but not limited to targeting the protein with a small molecule, a peptide, an aptamer, destabilizing domains, or other methods that can e.g., down-regulate the activity or enhance the rate of degradation of a gene product. In one preferred instance, a small molecule that binds e.g. an active site and inhibits the function of a target protein can be added to e.g., the cell culture media and thereby introduced into the cell. Alternatively, target protein function can be modulated by introducing e.g., a peptide into a cell that (for instance) prevents protein-protein interactions (see for instance, Shangary et. al., (2009) Annual Review of Pharmacology and Toxicology 49:223). Such peptides can be introduced into a cell by transfection or electroporation, or introduced via an expression construct. Alternatively, peptides can be introduced into cells by 1) adding (e.g., through conjugation) one or more moieties that facilitate cellular delivery, or 2) supercharging molecules to enhance self-delivery (Cronican, J. J. et al (2010) ACS Chem. Biol. 5(8):747-52). Techniques for expressing a peptide include, but are not limited to 1) fusion of the peptide to a scaffold, or 2) attachment of a signal sequence, to stabilize or direct the peptide to a position or compartment of interest, respectively.

43. It is understood and contemplated herein that some methods of increasing Rotaviral production can comprise administering siRNA, miRNA mimics, shRNA, or miRNA inhibitors to the media of a Rotavirus infected cell or cell line to produce a cell or cell line with decreased expression of a gene that inhibits Rotaviral production rather than starting the method with a cell or cell line so modified. In one aspect, disclosed herein are method of increasing Rotaviral production comprising infecting a cell or cell line with a Rotavirus and incubating the cell or cell line under conditions suitable for the production of the virus by the cells, wherein the medium comprises an RNA polynucleotide that inhibits expression of a coding region selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51. Also disclosed are method of increasing Rotavirus production wherein the RNA polynucleotide is an siRNA, miRNA mimics, shRNA, or miRNA inhibitor.

44. It is understood and herein contemplated that the timing of target gene modulation can vary. In some cases it is envisioned that gene modulation may occur prior to rotavirus infection. For instance, if the gene target of choice locks the cell in a particular phase of the cell cycle that is highly productive for rotavirus replication or RV antigen production, initiating gene modulation prior to viral infection may be beneficial. In other cases, it may be beneficial for rotavirus infection/replication or antigen production to be initiated prior to modulating the target gene of interest. For instance, if a particular host gene modulation event is essential at the later stages of viral replication or antigen production, but deleterious at the early stages, the inventors envision that gene modulation would be initiated after infection. In cases where two or more gene modulation events are required for optimized rotavirus or RV antigen production, some of the genes may be modified before viral infection while others are modified after viral infection. Regardless of the timing of gene modulation, multiple methods (including, for instance, applications of shRNA in conjunction with regulatable (e.g., Tet-sensitive promoter) can be employed to time the expression of gene modulation.

45. In one aspect, it is contemplated herein that any of the disclosed methods of increasing Rotaviral production disclosed herein can further comprise incubating the cells or cell line under conditions suitable for the production of the virus by the cells; and harvesting the virus produced by the cells.

46. In one aspect disclosed herein are methods of increasing Rotavirus production comprising infecting any cell or cell line disclosed herein with a Rotavirus. In another aspect disclosed are methods further comprising producing rotavirus vaccine in which cells having one or more genes or gene products modulated, are employed.

47. The compositions disclosed herein and the compositions necessary to perform the disclosed methods can be made using any method known to those of skill in the art for that particular reagent or compound unless otherwise specifically noted.

C. Compositions

48. Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 is disclosed and discussed and a number of modifications that can be made to a number of molecules including the ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 are discussed, specifically contemplated is each and every combination and permutation of ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

49. In one aspect the disclosed compositions can be cells or cell lines to be used in the disclosed methods of increasing Rotaviral production. In one aspect, disclosed herein are cells comprising reduced expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51.

50. As used herein, the term “gene” refers to a transcription unit and regulatory regions that are adjacent (e.g., located upstream and downstream), and operably linked, to the transcription unit. A transcription unit is a series of nucleotides that are transcribed into an RNA molecule. A transcription unit may include a coding region. A “coding region” is a nucleotide sequence that encodes an unprocessed preRNA (i.e., an RNA molecule that includes both exons and introns) that is subsequently processed to an mRNA. A transcription unit may encode a non-coding RNA. A non-coding RNA is an RNA molecule that is not translated into a protein. Examples of non-coding RNAs include microRNA. The boundaries of a transcription unit are generally determined by an initiation site at its 5′ end and a transcription terminator at its 3′ end. A “regulatory region” is a nucleotide sequence that regulates expression of a transcription unit to which it is operably linked. Nonlimiting examples of regulatory sequences include promoters, enhancers, transcription initiation sites, translation start sites, translation stop sites, transcription terminators, and poly(A) signals. A regulatory region located upstream of a transcription unit may be referred to as a 5′ UTR, and a regulatory region located downstream of a transcription unit may be referred to as a 3′ UTR. A regulatory region may be transcribed and be part of an unprocessed preRNA. The term “operably linked” refers to a juxtaposition of components such that they are in a relationship permitting them to function in their intended manner. It is understood and herein contemplated that wherein a particular gene is discussed herein, such as, for example ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51; also disclosed are any orthologs and variants of the disclosed gene for use in any composition or method disclosed herein.

51. It is recognized that any individual gene can be identified by any number of names and accession numbers. In many cases, genes in this document are identified by common gene names (e.g. dolichyldiphosphatase 1 (NAT9)) or accession numbers associated with the DNA sequence, mRNA sequence, or protein sequence (e.g., NM_015654). Furthermore it is recognized that for any reported DNA, RNA, or protein sequence, multiple sequence variants, splice variants or isoforms can be included in the databases. As the siRNAs used in this study are designed to suppress the expression of all variants/isoforms of a given gene, the gene targets identified in this document are intended to comprise all such variants/isoforms.

52. As disclosed herein, the disclosed cells or cell lines derived therefrom can comprise the reduced expression of any combination of one, two, three, four, five, six, seven, eight, nine, 10, 11, 12, 13, 14, 15, 16, 17, 18, 19, 20, 21, 22, 23, 24, 25, 30, 35, 40, 45, 50, 55, 60, 65, 70, 71, 72, 73, 74, 75, or all 76of the disclosed genes. For example, the cell can comprise reduced expression of NAT9 alone or in combination with any one, two, three, four, five, six, seven, eight, nine, or ten other of the selected genes. Thus, in one aspect, disclosed herein are cells comprising reduced expression of ZNF205; NEU2; NAT9; SVOPL; COQ9, BTN2A1, PYCR1, EP300, SEC61G; NDUFA9; RAD51AP1; COX20; MAPK6; WDR62; LRGUK; CDK6; KIAA1683; CRISP3; GRPR; DPH7; GEMIN8; KIAA1407; RFXAP; SMARRCA4; CCDC147; AACS; CDK9; C7ORF26; ZDHHC14; RNUT1; GAB1; EMC3; FAM96A; FAM36A; LOC55831; LOC136306; DEFB126; MGC955; EPHX2; SRGAP1; PPPSC; MET; SELM; TSPYL2; TSARG6; NDUFB2; PLAU; FLJ36888; ADORA2B; FLJ22875; HMMR; NRK, LRIT3; FLJ44691; GPR154; ZGPAT; DRD1; FLJ27505; EDG5; SNRNP40; HPRP8BP; GPA33; JDP2; FLJ20010; FOXJ1; SCT; CHD1L; SULT1C1; STN2; MRS2L; RAD51AP1; DPH7; CLPP; ZNF37; AP3B2; DEGS2; PIR; D2LIC; CNTF; PAM; MYH9; PRPF4; SLC4A11; LRRCC1; FZD9; GPR43; LTF; ARIH1; PIK3R3; PTGFRN; KIAA1764; C19ORF14; FLNA; FLJ32786; DKFZP434K046; C9ORF112; PIR51; NAT9 and NEU2; NAT9 and SVOPL; NAT9 and COQ9; NAT9 and NDUFA9; NAT9 and RAD51AP1; NAT9 and COX20; NAT9 and MAPK6; NAT9 and WDR62; NAT9 and LRGUK; NAT9 and CDK6; NAT9 and KIAA1683; NAT9 and CRISP3; NAT9 and GRPR; NAT9 and DPH7; NAT9 and GEMIN8; NAT9 and KIAA1407; NAT9 and RFXAP; NAT9 and SMARRCA4; NAT9 and CCDC147; NAT9 and AACS; NAT9 and CDK9; NAT9 and C7ORF26; NAT9 and ZDHHC14; NAT9 and RNUT1; NAT9 and GAB1; NAT9 and EMC3; NAT9 and FAM96A; NAT9 and FAM36A; NAT9 and LOC55831; NAT9 and LOC136306; NAT9 and DEFB126; NAT9 and MGC955; NAT9 and EPHX2; NAT9 and SRGAP1; NAT9 and PPPSC; NAT9 and MET; NAT9 and SELM; NAT9 and TSPYL2; NAT9 and TSARG6; NAT9 and NDUFB2; NAT9 and PLAU; NAT9 and FLJ36888; NAT9 and ADORA2B; NAT9 and FLJ22875; NAT9 and HMMR; NAT9 and NRK; NAT9 and FLJ44691; NAT9 and GPR154; NAT9 and ZGPAT; NAT9 and DRD1; NAT9 and FLJ27505; NAT9 and EDG5; NAT9 and SNRNP40; NAT9 and HPRP8BP; NAT9 and GPA33; NAT9 and JDP2; NAT9 and FLJ20010; NAT9 and FOXJ1; NAT9 and SCT; NAT9 and CHD1L; NAT9 and SULT1C1; NAT9 and STN2; NAT9 and MRS2L; NAT9 and RAD51AP1; NAT9 and DPH7; NAT9 and CLPP; NAT9 and ZNF37; NAT9 and AP3B2; NAT9 and COQ9; NAT9 and DEGS2; NAT9 and PIR; NAT9 and D2LIC; NAT9 and CNTF; NAT9 and PAM; NAT9 and MYH9; NAT9 and PRPF4; NAT9 and SLC4A11; NAT9 and LRRCC1; NAT9 and FZD9; NAT9 and GPR43; NAT9 and LTF; NAT9 and ARIH1; NAT9 and PIK3R3; NAT9 and PTGFRN; NAT9 and KIAA1764; NAT9 and C19ORF14; NAT9 and FLNA; NAT9 and FLJ32786; NAT9 and DKFZP434K046; NAT9 and C9ORF112; NAT9 and PIR51; NEU2 and SVOPL; NEU2 and COQ9; NEU2 and NDUFA9; NEU2 and RAD51AP1; NEU2 and COX20; NEU2 and MAPK6; NEU2 and WDR62; NEU2 and LRGUK; NEU2 and CDK6; NEU2 and KIAA1683; NEU2 and CRISP3; NEU2 and GRPR; NEU2 and DPH7; NEU2 and GEMIN8; NEU2 and KIAA1407; NEU2 and RFXAP; NEU2 and SMARRCA4; NEU2 and CCDC147 SVOPL and COQ9; SVOPL and NDUFA9; SVOPL and RAD51AP1; SVOPL and COX20; SVOPL and MAPK6; SVOPL and WDR62; SVOPL and LRGUK; SVOPL and CDK6; SVOPL and KIAA1683; SVOPL and CRISP3; SVOPL and GRPR; SVOPL and DPH7; SVOPL and GEMIN8; SVOPL and KIAA1407; SVOPL and RFXAP; SVOPL and SMARRCA4; SVOPL and CCDC147; COQ9 and NDUFA9; COQ9 and RAD51AP1; COQ9 and COX20; COQ9 and MAPK6; COQ9 and WDR62; COQ9 and LRGUK; COQ9 and CDK6; COQ9 and KIAA1683; COQ9 and CRISP3; COQ9 and GRPR; COQ9 and DPH7; COQ9 and GEMIN8; COQ9 and KIAA1407; COQ9 and RFXAP; COQ9 and SMARRCA4; COQ9 and CCDC147 ; NDUFA9 and RAD51AP1; NDUFA9 and COX20; NDUFA9 and MAPK6; NDUFA9 and WDR62; NDUFA9 and LRGUK; NDUFA9 and CDK6; NDUFA9 and KIAA1683; NDUFA9 and CRISP3; NDUFA9 and GRPR; NDUFA9 and DPH7; NDUFA9 and GEMIN8; NDUFA9 and KIAA1407; NDUFA9 and RFXAP; NDUFA9 and SMARRCA4; NDUFA9 and CCDC147 ; RAD51AP1 and COX20; RAD51AP1 and MAPK6; RAD51AP1 and WDR62; RAD51AP1 and LRGUK; RAD51AP1 and CDK6; RAD51AP1 and KIAA1683; RAD51AP1 and CRISP3; RAD51AP1 and GRPR; RAD51AP1 and DPH7; RAD51AP1 and GEMIN8; RAD51AP1 and KIAA1407; RAD51AP1 and RFXAP; RAD51AP1 and SMARRCA4; RAD51AP1 and CCDC147; COX20 and MAPK6; COX20 and WDR62; COX20 and LRGUK; COX20 and CDK6; COX20 and KIAA1683; COX20 and CRISP3; COX20 and GRPR; COX20 and DPH7; COX20 and GEMIN8; COX20 and KIAA1407; COX20 and RFXAP; COX20 and SMARRCA4; COX20 and CCDC147; MAPK6 and WDR62; MAPK6 and LRGUK; MAPK6 and CDK6; MAPK6 and KIAA1683; MAPK6 and CRISP3; MAPK6 and GRPR; MAPK6 and DPH7; MAPK6 and GEMIN8; MAPK6 and KIAA1407; MAPK6 and RFXAP; MAPK6 and SMARRCA4; MAPK6 and CCDC147; WDR62 and LRGUK; WDR62 and CDK6; WDR62 and KIAA1683; WDR62 and CRISP3; WDR62 and GRPR; WDR62 and DPH7; WDR62 and GEMIN8; WDR62 and KIAA1407; WDR62 and RFXAP; WDR62 and SMARRCA4; WDR62 and CCDC147; LRGUK and CDK6; LRGUK and KIAA1683; LRGUK and CRISP3; LRGUK and GRPR; LRGUK and DPH7; LRGUK and GEMIN8; LRGUK and KIAA1407; LRGUK and RFXAP; LRGUK and SMARRCA4; LRGUK and CCDC147; CDK6 and KIAA1683; CDK6 and CRISP3; CDK6 and GRPR; CDK6 and DPH7; CDK6 and GEMIN8; CDK6 and KIAA1407; CDK6 and RFXAP; CDK6 and SMARRCA4; CDK6 and CCDC147; KIAA1683 and CRISP3; KIAA1683 and GRPR; KIAA1683 and DPH7; KIAA1683 and GEMIN8; KIAA1683 and KIAA1407; KIAA1683 and RFXAP; KIAA1683 and SMARRCA4; KIAA1683 and CCDC147; CRISP3 and GRPR; CRISP3 and DPH7; CRISP3 and GEMIN8; CRISP3 and KIAA1407; CRISP3 and RFXAP; CRISP3 and SMARRCA4; CRISP3 and CCDC147; GRPR and DPH7; GRPR and GEMIN8; GRPR and KIAA1407; GRPR and RFXAP; GRPR and SMARRCA4; GRPR and CCDC147; DPH7 and GEMIN8; DPH7 and KIAA1407; DPH7 and RFXAP; DPH7 and SMARRCA4; DPH7 and CCDC147; GEMIN8 and KIAA1407; GEMIN8 and RFXAP; GEMIN8 and SMARRCA4; GEMIN8 and CCDC147; KIAA1407 and RFXAP; KIAA1407 and SMARRCA4; KIAA1407 and CCDC147; RFXAP and SMARRCA4; RFXAP and CCDC147; SMARRCA4 and CCDC147; ZNF205 and NEU2; ZNF205 and ZNF205, NAT9; ZNF205 and SVOPL; ZNF205 and COQ9; ZNF205 and NDUFA9; ZNF205 and RAD51AP1; ZNF205 and COX20; ZNF205 and MAPK6; ZNF205 and WDR62; ZNF205 and LRGUK; ZNF205 and CDK6; ZNF205 and KIAA1683; ZNF205 and CRISP3; ZNF205 and GRPR; ZNF205 and DPH7; ZNF205 and GEMIN8; ZNF205 and KIAA1407; ZNF205 and RFXAP; ZNF205 and SMARRCA4; ZNF205 and CCDC147; ZNF205 and AACS; ZNF205 and CDK9; ZNF205 and C7ORF26; ZNF205 and ZDHHC14; ZNF205 and RNUT1; ZNF205 and GAB1; ZNF205 and EMC3; ZNF205 and FAM96A; ZNF205 and FAM36A; ZNF205 and LOC55831; ZNF205 and LOC136306; ZNF205 and DEFB126; ZNF205 and MGC955; ZNF205 and EPHX2; ZNF205 and SRGAP1; ZNF205 and PPP5C; ZNF205 and MET; ZNF205 and SELM; ZNF205 and TSPYL2; ZNF205 and TSARG6; ZNF205 and NDUFB2; ZNF205 and PLAU; ZNF205 and FLJ36888; ZNF205 and ADORA2B; ZNF205 and FLJ22875; ZNF205 and HMMR; ZNF205 and NRK; ZNF205 and FLJ44691; ZNF205 and GPR154; ZNF205 and ZGPAT; ZNF205 and DRD1; ZNF205 and FLJ27505; ZNF205 and EDG5; ZNF205 and SNRNP40; ZNF205 and HPRP8BP; ZNF205 and GPA33; ZNF205 and JDP2; ZNF205 and FLJ20010; ZNF205 and FOXJ1; ZNF205 and SCT; ZNF205 and CHD1L; ZNF205 and SULT1C1; ZNF205 and STN2; ZNF205 and MRS2L; ZNF205 and RAD51AP1; ZNF205 and DPH7; ZNF205 and CLPP; ZNF205 and ZNF37; ZNF205 and AP3B2; ZNF205 and COQ9; ZNF205 and DEGS2; ZNF205 and PIR; ZNF205 and D2LIC; ZNF205 and CNTF; PAM; ZNF205 and MYH9; ZNF205 and PRPF4; ZNF205 and SLC4A11; ZNF205 and LRRCC1; ZNF205 and FZD9; ZNF205 and GPR43; ZNF205 and LTF; ZNF205 and ARIH1; ZNF205 and PIK3R3; ZNF205 and PTGFRN; ZNF205 and KIAA1764; ZNF205 and C19ORF14; ZNF205 and FLNA; ZNF205 and FLJ32786; ZNF205 and DKFZP434K046; ZNF205 and C9ORF112; ZNF205 and PIR51; ZNF205, NAT9 and NEU2; ZNF205, NAT9 and SVOPL; ZNF205, NAT9 and COQ9; ZNF205, NAT9 and NDUFA9; ZNF205, NAT9 and RAD51AP1; ZNF205, NAT9 and COX20; ZNF205, NAT9 and MAPK6; ZNF205, NAT9 and WDR62; ZNF205, NAT9 and LRGUK; ZNF205, NAT9 and CDK6; ZNF205, NAT9 and KIAA1683; ZNF205, NAT9 and CRISP3; ZNF205, NAT9 and GRPR; ZNF205, NAT9 and DPH7; ZNF205, NAT9 and GEMIN8; ZNF205, NAT9 and KIAA1407; ZNF205, NAT9 and RFXAP; ZNF205, NAT9 and SMARRCA4; ZNF205, NAT9 and CCDC147; ZNF205, NAT9 and AACS; ZNF205, NAT9 and CDK9; ZNF205, NAT9 and C7ORF26; ZNF205, NAT9 and ZDHHC14; ZNF205, NAT9 and RNUT1; ZNF205, NAT9 and GAB1; ZNF205, NAT9 and EMC3; ZNF205, NAT9 and FAM96A; ZNF205, NAT9 and FAM36A; ZNF205, NAT9 and LOC55831; ZNF205, NAT9 and LOC136306; ZNF205, NAT9 and DEFB126; ZNF205, NAT9 and MGC955; ZNF205, NAT9 and EPHX2; ZNF205, NAT9 and SRGAP1; ZNF205, NAT9 and PPP5C; ZNF205, NAT9 and MET; ZNF205, NAT9 and SELM; ZNF205, NAT9 and TSPYL2; ZNF205, NAT9 and TSARG6; ZNF205, NAT9 and NDUFB2; ZNF205, NAT9 and PLAU; ZNF205, NAT9 and FLJ36888; ZNF205, NAT9 and ADORA2B; ZNF205, NAT9 and FLJ22875; ZNF205, NAT9 and HMMR; ZNF205, NAT9 and NRK; ZNF205, NAT9 and FLJ44691; ZNF205, NAT9 and GPR154; ZNF205, NAT9 and ZGPAT; ZNF205, NAT9 and DRD1; ZNF205, NAT9 and FLJ27505; ZNF205, NAT9 and EDG5; ZNF205, NAT9 and SNRNP40; ZNF205, NAT9 and HPRP8BP; ZNF205, NAT9 and GPA33; ZNF205, NAT9 and JDP2; ZNF205, NAT9 and FLJ20010; ZNF205, NAT9 and FOXJ1; ZNF205, NAT9 and SCT; ZNF205, NAT9 and CHD1L; ZNF205, NAT9 and SULT1C1; ZNF205, NAT9 and STN2; ZNF205, NAT9 and MRS2L; ZNF205, NAT9 and RAD51AP1; ZNF205, NAT9 and DPH7; ZNF205, NAT9 and CLPP; ZNF205, NAT9 and ZNF37; ZNF205, NAT9 and AP3B2; ZNF205, NAT9 and COQ9; ZNF205, NAT9 and DEGS2; ZNF205, NAT9 and PIR; ZNF205, NAT9 and D2LIC; ZNF205, NAT9 and CNTF; ZNF205, NAT9 and PAM; ZNF205, NAT9 and MYH9; ZNF205, NAT9 and PRPF4; ZNF205, NAT9 and SLC4A11; ZNF205, NAT9 and LRRCC1; ZNF205, NAT9 and FZD9; ZNF205, NAT9 and GPR43; ZNF205, NAT9 and LTF; ZNF205, NAT9 and ARIH1; ZNF205, NAT9 and PIK3R3; ZNF205, NAT9 and PTGFRN; ZNF205, NAT9 and KIAA1764; ZNF205, NAT9 and C19ORF14; ZNF205, NAT9 and FLNA; ZNF205, NAT9 and FLJ32786; ZNF205, NAT9 and DKFZP434K046; ZNF205, NAT9 and C9ORF112; and ZNF205, NAT9 and PIR51.

53. The disclosed cells and cell lines derived therefrom can be any cell or cell line that can be stably infected with Rotavirus. In one aspect, the cells can be of mammalian origin (including, human, simian, porcine, bovine, equine, canine, feline, rodent (e.g., rabbit, rat, mouse, and guinea pig), and non-human primate) or avian including chicken, duck, ostrich, and turkey cells. It is further contemplated that the cell can be a cell of an established mammalian cell line including, but not limited to MA104 cells, VERO cells, Madin-Darby Canine Kidney (MDCK) cells, HEp-2 cells, HeLa cells, HEK293 cells, MRC-5 cells, WI-38 cells, EB66, and PER C6 cells.

54. In one aspect, the cells or cell lines disclosed herein can have reduced expression or copy number of genes, mRNA, or proteins or reduced protein activity that inhibits Rotaviral production. Reduction in expression can be at least a 1, 2, 3, 4, 5, 6, 7, 8, 9, 10, 15, 20, 25, 30, 35, 40, 45, 50, 55, 60, 65, 70, 75, 80, 85, 86, 87, 88, 89, 90, 91, 92, 93, 94, 95, 96, 97, 98, 99, or 100% reduction of the gene expression, mRNA translation, protein expression, or protein activity relative to a control. For example, disclosed herein are cells and/or cell lines comprising at least 1, 2, 3, 4, 5, 6, 7, 8, 9, 10, 15, 20, 25, 30, 35, 40, 45, 50, 55, 60, 65, 70, 75, 80, 85, 86, 87, 88, 89, 90, 91, 92, 93, 94, 95, 96, 97, 98, 99, or 100% reduction of the expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes relative to a control.

55. It is further understood that one way of referring to a reduction rather than the percentage reduction is as a percentage of the control expression or activity. For example, a cell with at least a 15% reduction in the expression of a particular gene relative to a control would also be a gene with expression that is less than or equal to 85% of the expression of the control. Accordingly, in one aspect, disclosed herein are cells or cell lines wherein the gene expression, mRNA expression, protein expression, or protein activity is less than or equal to 95, 90, 89, 88, 87, 86, 85, 84, 83, 82, 81, 80, 75, 70, 65, 60, 55, 50, 45, 40, 35, 30, 25, 20, 15, 10, 9, 8, 7, 6, 5, 4, 3, 2, 1% of a control. Thus, disclosed herein are cells or cell lines comprising less than or equal to 95, 90, 89, 88, 87, 86, 85, 84, 83, 82, 81, 80, 75, 70, 65, 60, 55, 50, 45, 40, 35, 30, 25, 20, 15, 10, 9, 8, 7, 6, 5, 4, 3, 2, 1% ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 relative to a control. For example, disclosed herein are cell comprising less than or equal to 85% reduction of the expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 relative to a control.

56. It is understood and herein contemplated that the reduced expression can be achieved by any means known in the art including techniques that manipulate genomic DNA, messenger and/or non-coding RNA and/or proteins including but not limited to endogenous or exognenous control elements (e.g., siRNA, shRNA, small molecule inhibitor, and antisense oligonucleotide) and mutations in or directly targeting the coding region of the gene, mRNA, or protein or a control element or mutation in a regulator region operably linked to the gene, mRNA, or protein. As such, the technologies or mechanisms that can be employed to modulate a gene of interest include but are not limited to 1) technologies and reagents that target genomic DNA to result in an edited genome (e.g., homologous recombination to introduce a mutation such as a deletion into a gene, zinc finger nucleases, meganucleases, transcription activator-like effectors (e.g., TALENs), triplexes, mediators of epigenetic modification, and CRISPR and rAAV technologies), 2) technologies and reagents that target RNA (e.g. agents that act through the RNAi pathway, antisense technologies, ribozyme technologies), and 3) technologies that target proteins (e.g., small molecules, aptamers, peptides, auxin- or FKBP-mediated destabilizing domains, antibodies).

57. In one embodiment for targeting DNA, gene modulation is achieved using zinc finger nucleases (ZFNs). Synthetic ZFNs are composed of a custom designed zinc finger binding domain fused with e.g. a Fokl DNA cleavage domain. As these reagents can be designed/engineered for editing the genome of a cell, including, but not limited to, knock out or knock in gene expression, in a wide range of organisms, they are considered one of the standards for developing stable engineered cell lines with desired traits. Meganucleases, triplexes, TALENs, CRISPR, and recombinant adeno-associated viruses have similarly been used for genome engineering in a wide array of cell types and are viable alternatives to ZFNs. The described reagents can be used to target promoters, protein-encoding regions (exons), introns, 5′ and 3′ UTRs, and more.

58. Another embodiment for modulating gene function utilizes the cell's endogenous or exogenous RNA interference (RNAi) pathways to target cellular messenger RNA. In this approach, gene targeting reagents include small interfering RNAs (siRNA) as well as microRNAs (miRNA). These reagents can incorporate a wide range of chemical modifications, levels of complementarity to the target transcript of interest, and designs (see U.S. Pat. No. 8,188,060) to enhance stability, cellular delivery, specificity, and functionality. In addition, such reagents can be designed to target diverse regions of a gene (including the 5′ UTR, the open reading frame, the 3′ UTR of the mRNA), or (in some cases) the promoter/enhancer regions of the genomic DNA encoding the gene of interest. Gene modulation (e.g., knockdown) can be achieved by introducing (into a cell) a single siRNA or miRNA or multiple siRNAs or miRNAs (i.e., pools) targeting different regions of the same mRNA transcript. Synthetic siRNA/miRNA delivery can be achieved by any number of methods including but not limited to 1) self-delivery (US Patent Application No 2009/0280567A1), 2) lipid-mediated delivery, 3) electroporation, or 4) vector/plasmid-based expression systems. An introduced RNA molecule may be referred to as an exogenous nucleotide sequence or polynucleotide.

59. Another gene targeting reagent that uses RNAi pathways includes exogenous small hairpin RNA, also referred to as shRNA. shRNAs delivered to cells via e.g., expression constructs (e.g., plasmids, lentiviruses) have the ability to provide long term gene knockdown in a constitutive or regulated manner, depending upon the type of promoter employed. In one preferred embodiment, the genome of a lentiviral particle is modified to include one or more shRNA expression cassettes that target a gene (or genes) of interest. Such lentiviruses can infect a cell intended for vaccine production, stably integrate their viral genome into the host genome, and express the shRNA(s) in a 1) constitutive, 2) regulated, or (in the case where multiple shRNA are being expressed) constitutive and regulated fashion. In this way, cell lines having enhanced Rotavirus production capabilities can be created. It is worth noting, that approaches that use siRNA or shRNA have the added benefit in that they can be designed to target individual variants of a single gene or multiple closely related gene family members. In this way, individual reagents can be used to modulate larger collections of targets having similar or redundant functions or sequence motifs. The skilled person will recognize that lentiviral constructs can also incorporate cloned DNA, or ORF expression constructs.

60. In another embodiment for modulating gene function, gene suppression can be achieved by large scale transfection of cells with miRNA mimics or miRNA inhibitors introduced into the cells.

61. In another embodiment, modulation takes place at the protein level. By example, knockdown of gene function at the protein level can be achieved by a number of means including but not limited to targeting the protein with a small molecule, a peptide, an aptamer, destabilizing domains, or other methods that can e.g., down-regulate the activity or enhance the rate of degradation of a gene product. In one preferred instance, a small molecule that binds e.g. an active site and inhibits the function of a target protein can be added to e.g., the cell culture media and thereby introduced into the cell. Alternatively, target protein function can be modulated by introducing e.g., a peptide into a cell that (for instance) prevents protein-protein interactions (see for instance, Shangary et. al., (2009) Annual Review of Pharmacology and Toxicology 49:223). Such peptides can be introduced into a cell by transfection or electroporation, or introduced via an expression construct. Alternatively, peptides can be introduced into cells by 1) adding (e.g., through conjugation) one or more moieties that facilitate cellular delivery, or 2) supercharging molecules to enhance self-delivery (Cronican, J. J. et al (2010) ACS Chem. Biol. 5(8):747-52). Techniques for expressing a peptide include, but are not limited to 1) fusion of the peptide to a scaffold, or 2) attachment of a signal sequence, to stabilize or direct the peptide to a position or compartment of interest, respectively.

62. As discussed above, the compositions and methods disclosed herein fully contemplate cell lines comprising the cells described herein. As used herein, the term “cell line” refers to a clonal population of cells that are able to continue to divide and not undergo senescence. The cell(s) can be derived from any number of sources including mammalian (including but not limited to human, non-human primate, hamster, dog), avian (e.g., chicken, duck), insect, and more. The cell lines contemplated herein can also be modified versions of existing cell lines including but not limited to MA104 cells, VERO cells, Madin-Darby Canine Kidney (MDCK) cells, HEp-2 cells, HeLa cells, HEK293 cells, MRC-5 cells, WI-38 cells, EB66, and PER C6 cells. Preferably, the modified genes enhance RV antigen production or production of rotavirus strains used to produce RV vaccines. Preferably, the cell line and the rotavirus or RV antigen are employed in rotavirus vaccine production. Thus in one aspect disclosed herein are cell lines (including engineered cell lines) comprising a cell; wherein the cell comprises decreased expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes relative to a control.

63. The original screen for genes that enhanced rotavirus production took place in a MA104 cell line. MA104 cells are derived from monkey kidneys (Macaca mulatta in origin) and for this reason, the original screen identified Macaca monkey genes that when modulated, enhance rotavirus production. As described in the Examples section below, validation for the rotavirus hits utilized VERO cells which are derived from African Green Monkey (Chlorocebus). As hits identified in the primary screen also increase rotavirus titers in VERO cells, an additional embodiment includes a list of genes that are orthologs of those identified in the primary screen (Table I). Such orthologs can be modulated in human or non-human cells or cell lines to increase rotavirus or rotavirus antigen production.

64. Another embodiment includes knockout animals (e.g., knockout mice) having one or more of the genes identified in the Table 1 or 3 below modified to enhance rotavirus replication. For example, disclosed herein are knockout animals having one or more of the genes selected from the group comprising ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 modified to enhance rotavirus replication.

### 1. NUCLEIC ACIDS

65. There are a variety of molecules disclosed herein that are nucleic acid based, including for example the nucleic acids that encode, for example ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51, or any of the nucleic acids disclosed herein for making ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 knockouts, knockdowns, variants, mutants, or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U or variants thereof. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

66. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

67. A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modifcation, such as 2′-O-methoxyethyl, to achieve unique properties such as increased duplex stability.

68. Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modiifcations also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

69. Other modifications at the 2′ position include but are not limted to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar.

70. Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

71. It is understood that nucleotide analogs need only contain a single modification, but may also contain multiple modifications within one of the moieties or between different moieties.

72. Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

73. Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones;formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

74. It is also understood in a nucleotide substitute that both the sugar and the phosphate moieties of the nucleotide can be replaced, by for example an amide type linkage (aminoethylglycine) (PNA).

75. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

76. A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

77. A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

78. There are a variety of sequences ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXE, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C190RF14, FLNA, FLJ32786, DKFZP434K046, C9ORF112, and/or PIR51, or any of the nucleic acids disclosed herein for making ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51, all of which are encoded by nucleic acids or are nucleic acids. The sequences for the human analogs of these genes, as well as other analogs, and alleles of these genes, and splice variants and other types of variants, are available in a variety of protein and gene databases, including Genbank. Those of skill in the art understand how to resolve sequence discrepancies and differences and to adjust the compositions and methods relating to a particular sequence to other related sequences. Primers and/or probes can be designed for any given sequence given the information disclosed herein and known in the art.

c) Functional Nucleic Acids

79. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

80. Functional nucleic acid molecules can interact with any macromolecule, such as DNA, RNA, polypeptides, or carbohydrate chains. Thus, functional nucleic acids can interact with the mRNA of any of the disclosed nucleic acids, such as ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51, or the genomic DNA of any of the disclosed nucleic acids, such as ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51, or they can interact with the polypeptide encoded by any of the disclosed nucleic acids, such as ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51. Often functional nucleic acids are designed to interact with other nucleic acids based on sequence complementarity between the target molecule and the functional nucleic acid molecule. In other situations, the specific recognition between the functional nucleic acid molecule and the target molecule is not based on sequence complementarity between the functional nucleic acid molecule and the target molecule, but rather is based on the formation of tertiary structure that allows specific recognition to take place.

81. Antisense molecules are designed to interact with a target nucleic acid molecule through either canonical or non-canonical base pairing. The interaction of the antisense molecule and the target molecule is designed to promote the destruction of the target molecule through, for example, RNAse mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹².

82. Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline, as well as large molecules, such as reverse transcriptase and thrombin. Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the kd with a background binding molecule. It is preferred when doing the comparison for a polypeptide for example, that the background molecule be a different polypeptide.

83. Ribozymes are nucleic acid molecules that are capable of catalyzing a chemical reaction, either intramolecularly or intermolecularly. Ribozymes are thus catalytic nucleic acid.

It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo.

Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence.

84. Triplex forming functional nucleic acid molecules are molecules that can interact with either double-stranded or single-stranded nucleic acid. When triplex molecules interact with a target region, a structure called a triplex is formed, in which there are three strands of

DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹².

85. External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate.

### 2. NUCLEIC ACID DELIVERY

86. In the methods described above which include the administration and uptake of exogenous DNA or RNA into the cells of a subject or cell (i.e., gene transduction or transfection), the disclosed nucleic acids can be in the form of naked DNA or RNA, or the nucleic acids can be in a vector for delivering the nucleic acids to the cells, whereby the DNA or

RNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

87. As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

a) Delivery of the Compositions to Cells

88. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, include chemical transfectants, or physico-mechanical methods such as electroporation and direct diffusion of DNA. Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

b) Nucleic Acid Based Delivery Systems

89. Transfer vectors can be any nucleotide construction used to deliver genes into cells (e.g., a plasmid), or as part of a general strategy to deliver genes, e.g., as part of recombinant retrovirus or adenovirus.

90. Viral vectors are, for example, Adenovirus, Adeno-associated virus, Herpes virus, Lenti virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature.

91. Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

c) Retroviral Vectors

92. A retrovirus is an animal virus belonging to the virus family of Retroviridae, including any types, subfamilies, genus, or tropisms (e.g., Lentivirus). Retroviral vectors, in general, are described by Verma, I.M., Retroviral vectors for gene transfer.

93. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

94. Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

d) Adenoviral Vectors

95. The construction of replication-defective adenoviruses has been described. The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites. Recombinant adenoviruses achieve gene transduction by binding to specific cell surface receptors, after which the virus is internalized by receptor-mediated endocytosis, in the same manner as wild type or replication-defective adenovirus

96. A viral vector can be one based on an adenovirus which has had the E1 gene removed and these virons are generated in a cell line such as the CHO and HEK293 cell lines. In another preferred embodiment both the E1 and E3 genes are removed from the adenovirus genome.

e) Adeno-Associated Viral Vectors

97. Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

98. In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

99. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

100. The disclosed vectors thus provide DNA molecules which are capable of integration into a mammalian chromosome without substantial toxicity.

101. The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

f) Large Payload Viral Vectors

102. Molecular genetic experiments with large human herpesviruses have provided a means whereby large heterologous DNA fragments can be cloned, propagated and established in cells permissive for infection with herpesviruses. These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

103. Other useful systems include, for example, replicating and host-restricted non-replicating vaccinia virus vectors.

g) Non-Nucleic Acid Based Systems

104. The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

105. Thus, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Furthermore, the compound can be administered as a component of a microcapsule that can be targeted to specific cell types, such as macrophages, or where the diffusion of the compound or delivery of the compound from the microcapsule is designed for a specific rate or dosage.

106. In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

107. The materials may be in solution, suspension (for example, incorporated into microparticles, liposomes, or cells). These may be targeted to a particular cell type via antibodies, receptors, or receptor ligands. These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, and type of ligand, ligand valency, and ligand concentration.

108. Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

109. Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### 3. EXPRESSION SYSTEMS

110. The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

111. Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

112. Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

113. In certain embodiments the promoter and/or enhancer region can act as a constitutive promoter and/or enhancer to maximize expression of the region of the transcription unit to be transcribed. Thus, in one embodiment disclosed herein are recombinant cells comprising one or more microRNA and at least one immunoglobulin encoding nucleic acid wherein the expression of the microRNa is constitutive. In such circumstances, the microRNA can be operationally linked to the constitutive promoter. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

114. In other embodiments, the promoter and/or enhancer region can act as an inducible promoter and/or enhancer to regulate expression of the region of the transcript to be transcribed. The promoter and/or enhancer may be specifically activated either by light, temperature, or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs. Other examples of inducible promoter systems include but are not limited to GAL4 promoter, Lac promoter, Cre recombinase (such as in a cre-lox inducible system), metal-regulated systems such as metallothionein, Flp-FRT recombinase, alcohol dehydrogenase I (alcA) promoter, and steroid regulated systems, such as, estrogen receptor (ER) and glucocorticoid receptor (GR). Inducible systems can also comprise inducible stem loop expression systems. Thus, in one embodiment disclosed herein are recombinant cells comprising one or more microRNA and at least one immunoglobulin encoding nucleic acid wherein the expression of the microRNA is inducible.

115. It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

116. Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

117. The viral vectors can include nucleic acid sequence encoding a marker product.

This marker product is used to determine if the gene has been delivered to the cell and once delivered is being expressed. Preferred marker genes are the E. Coli lacZ gene, which encodes ß-galactosidase, and green fluorescent protein.

118. In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

119. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin,. The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### 4. SEQUENCE SIMILARITIES

120. It is understood that as discussed herein the use of the terms “homology” and “identity” mean the same thing as “similarity.” Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

121. In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

122. Another way of calculating homology can be performed by published algorithms Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

123. It is understood that any of the methods typically can be used and that in certain instances the results of these various methods may differ, but the skilled artisan understands if identity is found with at least one of these methods, the sequences would be said to have the stated identity, and be disclosed herein.

124. For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

125. Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

126. The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

127. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 5. EXAMPLE 1

a) Methods

128. Both MA 104 and Vero cells were maintained in Dulbecco's modified Eagle's medium (DMEM, Thermo Fisher Scientific, Cat. # Sh30243.01) supplemented with 10% calf serum (HyClone, Cat. # Sh30396.03) and containing 1% penicillin-streptomycin (Cellgro, Cat. #30-004-CI) during propagation. The, MA-104 cell line was used for primary screening. Vero cells (African Green Monkey kidney cells) were received from the Centers for Disease Control and Prevention, Atlanta.

129. For siRNA transfections, On-TARGETplus (OTP)-siRNAs (Dharmacon Products) were reverse transfected into MA 104 cells at a final siRNA concentration of 50 nM in 0.4% DharmaFECT4 (DF4, Dharmacon) with 14,000 MA 10⁴ cells/per well in a 96-well plate. To achieve this, DF4 was first diluted in serum-free medium (OPTI-MEM) for 5 minutes. This material was then added to 96-well culture plates containing 5 μl of a 1 μM siRNA solution. The DF4-siRNA mixture was then incubated for 20 minutes (room temperature) prior to the addition of cells in Dulbecco's Modified Eagle's Medium supplemented with 10% calf serum. Transfected cells were then cultured for 48 hrs at 37° C., 5% CO₂. Subsequently, the media was removed, wells were washed 3× in 1× PVBS, and cells were infected at an MOI of 0.1 using a RV3 strain of rotavirus that was diluted in DMEM containing 2% calf serum and 1% penicillin-streptomycin. For the primary screen, plates containing the virus-infected MA 104 cells were removed from the culture incubator 24 hrs after virus infection and fixed for an FFN assay. Each plate also contained multiple controls including: 1) siTox (Dharmacon), 2) siNon-targeting control (Dharmacon), 3) rotavirus-specific siRNAs as a positive control targeting RV3 NSP2, and 4) a mock control.

130. For validation experiments a similar protocol that utilized Vero P cells was followed. Briefly, OTP-siRNAs were reverse transfected into Vero P cells at a final siRNA concentration of 50nM in 0.4% DF4, with 7,500 cells/well. As described above, DF4 was diluted in serum-free OPTI-MEM for 5 minutes prior to adding the transfection reagent to 96-well culture plates containing Sul of a 1μM siRNA solution. The DF4-siRNA cocktail was then incubated for 20 minutes at room temperature prior to addition of Vero P cells in DMEM supplemented with 10% calf serum. Transfected cells were then cultured for 48 hrs at 37° C., 5% CO₂. The media was then removed and cells were infected at an MOI of 0.2 using the RV3 rotavirus strain diluted in DMEM containing 2% calf serum and 1% penicillin-streptomycin. The plates containing the virus-infected Vero P cells were removed from culture 48 hrs later and assayed as previously described.

### (1) Silencing Reagents siRNAs

131. The ON-TARGETplus siRNA (OTP-siRNA) library (Dharmacon) was used for the primary RNAi screen. OTP silencing reagents are provided as a pool of siRNA targeting each gene. Each pool contains 4 individual siRNAs targeting different regions of the open reading frame (ORF). siRNA pools are designed to target all splice variants of the genes, thus in cases where a particular Accession Number is identified, it is understood that all variants of that gene are targeted by the siRNA.

132. For deconvolution validation experiments, each of the siRNA comprising the OTP pool was tested individually to determine if two or more siRNA generated the observed phenotype.

### (2) Cell Viability Assay and Cell Proliferation Assay

133. To examine whether the transfection of siRNA negatively affected screen results by inducing cellular toxicity, the TOXILIGHTTM bioassay (LONZA Inc.) was incorporated in both the primary screen and hit validation studies. TOXILIGHT™ is a non-destructive bioluminescent cytotoxicity assay designed to measure toxicity in cultured mammalian cells and cell lines. The method, which quantitatively measures the release of adenylate kinase (AK) from damaged cells, was employed by assessing the culture supernatant 48 hours after siRNA transfection. To examine whether knockdown of the identified target genes affected cell growth, the CELLTITER 96® Assay (PROMEGA Inc., Kit cat. # G3580) was employed to determine viable cell numbers. The CELLTITER 96® Assay has been shown to provide greater signal sensitivity and stability compared to other MTT assays. In the studies provided herein, 48 or 72 hrs after siRNA transfection, the substrate for the CELLTITER 96® Assay assay was added directly to the culture plates. Following a 4 hr incubation at 37° C., the culture absorbance was measured at OD495 nm.

### (3) The Rotavirus FFN Assay

134. Two days post siRNA transfection, MA104 cells were infected with an activated rotavirus for 24 hours. Subsequently the supernatant was removed and cells were fixed prior to performing an immunofluorescent ELISA. For immunofluorescent staining, fixed plates were washed 2× with PBS and then blocked for 1 hr at room temperature (0.05% PBST containing BSA). The primary polyclonal rabbit anti-Rotavirus antibody (Rab A-SA11, Australia) in blocking solution was added (50 ul per well) for 1 hr at room temperature. Afterwards the primary solution was removed and the plates were washed (4× with 0.05% PBST) followed by the addition of a fluorescently labeled secondary antibody (goat/Anti-rabbit Alexa 488, 50 ul per well,) for 1 hr at room temperature. Plates were then washed (2× with PBST and 2× with PBS) and read with a Beckman Coulter Paradigm spectrophotometer at 488 wavelength. For the initial screen fluorescent readings were normalized and hits showing a Z-score of 3.0 or greater were selected for validation.

### (4) Data Analysis Methods Used in the HTS Screening

135. In the current MA104 siRNA screen, the positive control siRNA targeting the RV3 {RV3-specific (NSP2-842)}, and the negative control (non-targeting siRNA) were clearly distinguishable from each other in all of the 96-well plates transfected with siRNAs. siTOX, a cytotoxic sequence, served as indicator for transfection efficiency and a mock control was used as background normalization. Quality control was assessed using Z′-factor where a Z′-factor scores between 0.5 and 1.0 is indicative of a highly robust assay whereas scores between 0 and 0.5 are deemed acceptable (see Zhang et al., 1999). Hits with a Z-score ≥3.0 SD were moved into the second phase of the program, validation.

### 6. EXAMPLE 2

### Primary Screen Results

136. Using the techniques described above, >18,200 genes from the human genome, including genes from the protease, ion channel, ubiqutin, kinase, phosphatase, GPCR, and drug target collections were screened to identify gene knockdown events that enhanced rotavirus replication. FIG. 1 shows a plot of the Z-scores obtained from the primary screen. As indicated, only a small fraction of the total gene knockdown events gave scores equal to or greater than 2.9 standard deviation (SD) from the mean (Table 1. 76 genes, 0.41% of the total number of genes screened). The genes contained in this collection were distributed across multiple functional families (kinases, proteases, phosphatases, etc.) and included a significant number of targets not previously identified as “antiviral”.

TABLE I
List of genes that when silenced increase rotavirus antigen/virus
production. Accession numbers retrieved from PubMed.

| Gene name | Z-score | Accession No. |
|---|---|---|
| NAT9 | 5.32 | NM_015654 |
| SVOPL | 4.91 | NM_001139456 |
| EMC3 | 4.59 | NM_018447 |
| AACS | 4.41 | NM_023928 |
| CDK9 | 4.17 | NM_001261 |
| C7ORF26 | 4.16 | NM_024067 |
| ZDHHC14 | 4.02 | NM_024630 |
| RNUT1 | 3.98 | NM_005701 |
| CDK6 | 3.97 | NM_001259 |
| GAB1 | 3.96 | NM_207123 |
| COX20 | 3.96 | NM_198076 |
| DEFB126 | 3.91 | NM_030931 |
| MGC955 | 3.89 | BC001508 |
| EPHX2 | 3.84 | NM_001979 |
| SRGAP1 | 3.79 | NM_020762 |
| MAPK6 | 3.77 | NM_002748 |
| PPP5C | 3.75 | NM_006247 |
| KIAA1407 | 3.74 | NM_020817 |
| MET | 3.62 | NM_001127500 |
| SELM | 3.56 | NM_080430 |
| TSPYL2 | 3.55 | NM_022117 |
| TSARG6 | 3.55 | AY138810 |
| NDUFB2 | 3.53 | NM_004546 |
| PLAU | 3.53 | NM_002658 |
| FAM96A | 3.53 | NM_032231 |
| ADORA2B | 3.52 | NM_000676 |
| HMMR | 3.49 | NM_001142556 |
| NRK | 3.48 | NM_198465 |
| FLJ44691 | 3.44 | CM000255.1 |
| LRIT3 | 3.44 | NM_198506.4 |
| GPR154 | 3.43 | BK005424 |
| CRISP3 | 3.43 | NM_006061 |
| ZGPAT | 3.43 | NM_032527 |
| DRD1 | 3.43 | NM_000794 |
| KIAA1683 | 3.39 | NM_001145304 |
| FLJ27505 | 3.35 | AK131015 |
| EDG5 | 3.34 | AF034780 |
| SNRNP40 | 3.33 | NM_004814 |
| GPA33 | 3.32 | NM_005814 |
| CCDC147 | 3.31 | NM_001008723 |
| RFXAP | 3.31 | NM_000538 |
| LRGUK | 3.30 | NM_144648 |
| JDP2 | 3.29 | NM_130469 |
| FLJ20010 | 3.29 | AK000017 |
| FOXJ1 | 3.28 | NM_001454 |
| GRPR | 3.28 | NM_005314 |
| SCT | 3.25 | NM_021920 |
| CHD1L | 3.22 | NM_004284 |
| NDUFA9 | 3.22 | NM_005002 |
| SULT1C1 | 3.19 | AF186256 |
| STN2 | 3.16 | NM_033104 |
| MRS2L | 3.15 | NM_001286264 |
| RAD51AP1 | 3.15 | NM_001130862 |
| DPH7 | 3.10 | NM_138778 |
| CLPP | 3.09 | Z50853 |
| ZNF537 | 3.09 | NM_020856 |
| AP3B2 | 3.08 | NM_001278512 |
| COQ9 | 3.08 | NM_020312 |
| DEGS2 | 3.08 | NM_206918 |
| PIR | 3.08 | NM_003662 |
| D2LIC | 3.07 | NM_016008 |
| CNTF | 3.05 | NM_000614 |
| PAM | 3.05 | NM_000919 |
| WDR62 | 3.04 | NM_001083961 |
| MYH9 | 3.02 | NM_002473 |
| PRPF4 | 3.02 | NM_004697 |
| SLC4A11 | 3.01 | NM_001174090 |
| LRRCC1 | 3.01 | NM_033402 |
| FZD9 | 3.00 | NM_003508 |
| GPR43 | 2.99 | NM_005306 |
| GEMIN8 | 2.98 | NM_001042480 |
| LTF | 2.98 | NM_002343 |
| SMARCA4 | 2.97 | NM_001128849 |
| ARIH1 | 2.95 | NM_005744 |
| PIK3R3 | 2.94 | NM_003629 |
| PTGFRN | 2.94 | NM_020440 |
| HSPA5BP1 | 2.57 | NM_178031.2 |
| ZDHHC16 | 2.15 | NM_001287803.1 |

Table provides gene symbol, primary screen SD value, and NCIB nucleotide accession number obtained from the NCBI resources database

### 7. EXAMPLE 3

### Validation of Effects of Gene Knockdown in Vero Cells

137. To determine whether the gene knockdown events identified in the primary screen enhanced RV3 production in a vaccine manufacturing cell line, the studies were repeated in Vero cells. Briefly, Vero cells transfected with pools of siRNA targeting each of the 76 genes were infected with RV3 and supernatants were then retrieved and assessed by ELISA. FIG. 2 shows the top 20 hits in Vero and demonstrates that hits identified in the primary MA 104 screen induce similar phenotypes in a second cell line (Vero).

### 8. EXAMPLE 4

### Pool Deconvolution Validation Studies

138. As an additional step in validation, primary screen hits that increased RV production in Vero cells were assessed to determine whether they were true positives or false positives. It is well known in the field of RNAi research that siRNAs can induce false positive phenotypes. One method of demonstrating that a hit is a true positive is to demonstrate that multiple individual siRNAs targeting different positions in the target gene induce the same “increase in virus titer” phenotype. To assess this, the pools of siRNA used in the primary screen were broken into four individual reagents and retested in Vero cells. To achieve this, Vero cells transfected with individual siRNA were infected with the RV3 virus and culture supernatants were assessed for the presence of virus using an ELISA.

139. Results from the top 20 hits from the Vero studies (Example 3) are presented in Table 3. In all cases, two or more siRNAs induced the “increase in antigen/virus” phenotype for each of the genes being studied. These findings, combined with the observation that KD of these genes increases antigen/virus production in two different cell types (MA 104 and Vero) strongly suggests these targets are true positives.

TABLE 3
siRNA pool deconvolution studies on top 20 gene targets.

| Gene name | # of siRNAs |
|---|---|
| NEU2 | 4 |
| NAT9 | 3 |
| SVOPL | 2 |
| COQ9 | 3 |
| NDUFA9 | 3 |
| RAD51AP1 | 3 |
| COX20 | 2 |
| MAPK6 | 3 |
| WDR62 | 4 |
| LRGUK | 4 |
| CDK6 | 3 |
| KIAA1683 | 3 |
| CRISP3 | 3 |
| GRPR | 2 |
| DPH7 | 3 |
| GEMIN8 | 2 |
| KIAA1407 | 2 |
| RFXAP | 3 |
| SMARRCA4 | 4 |
| CCDC147 | 4 |

Gene symbol and number of individual siRNAs that increase production are reported.

### 9. EXAMPLE 5

### Assessment of Gene Knockdown Levels in Vero Cells

140. Quantitative PCR was performed on the top ten hits identified in Example 4 to determine whether a correlation existed between the increase in antigen/virus phenotype and suppression of gene expression. To achieve this, Vero cells were transfected with siRNA pools targeting each of the genes of interest. Subsequently, RNA was isolated from each of the cultures and transcripts were quantitated by standard quantitative PCR methods.

141. As shown in FIG. 3, introduction of siRNAs suppressed the expression of each of the genes by as much as 90%. These results provide a strong correlation between gene suppression and increases in RV3 antigen/virus production.

### 10. EXAMPLE 6

### Assessment of Gene Knockout on Viral Replication of Various Rotaviral Strains

To assess the effect of gene knockouts vero cells, or cell lines comprising a knockout of the WDR62 gene or LRGUK gene were infected with Rotarix at an MOI of 0.2 for 3 days or 5 days. At 3 days post infection with Rotarix (FIG. 5A), the cells comprising a knockout of either the WDR62 or LRGUK gene showed a significant increase in the number of infected cells relative to Vero control cells. WDR62 knockout cells had approximately 10-fold greater production than cells comprising a knockout of the LRGUK gene. By 5 days post-infection the amount of rotalviral infected cells in the LRGUK knockout cells had increased more rapidly than in WDR62 cells such that the number of infected cells in the WDR62 knockouts was now less than 2-fold greater than in the LRGUK knockout cells. This data was confirmed using a rabbit anti-RV antigen and measuring viral levels in the sera at 3 days (FIG. 6A) and 5 days (FIG. 6B) post infection. Experiments were repeated with two other rotaviral strains CD9 (FIGS. 7 and 8) and 116E (FIGS. 9 and 10) having near comparable results.

What is claimed is:

1. A method of increasing Rotavirus production of one or more Rotaviruses comprising infecting a cell with a Rotavirus; wherein the cell comprises reduced expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51.

2. The method of claim 1, wherein the gene expression is reduced at least 15% relative to a control.

3. The method of claim 1, wherein the reduction occurs through a mutation in a regulator region operably linked to the coding region for ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes.

4. The method of claim 1, wherein the reduction in gene expression occurs through an exogenous control element.

5. The method of claim 4, wherein the exogenous control element is a siRNA, shRNA, small molecule inhibitor, or antisense polynucleotide.

6. The method of claim 5, wherein the exogenous control element targets the coding region for ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes.

7. The method of claim 1, wherein the reduction of gene expression occurs through insertion, substitution, or deletion of a portion of the coding region using a nuclease selected from the group consisting of zinc finger nucleases (ZFNs), meganucleases, transcription activator-like effectors (e.g., TALENs), triplexes, mediators of epigenetic modification, CRISPR and rAAV.

8. The method of claim 1, wherein the Rotavirus is selected from at least one species of Rotavirus A, Rotavirus B, Rotavirus C, Rotavirus D, Rotavirus E, Rotavirus F, Rotavirus G, or Rotavirus H; preferably the rotavirus is G1P7, G2,P7, G3P7, G4P7, G6P1A, G9 variants, RotaTeq strain, Rotarix strain, CDC9 strain, 116E strain, or RV3-BB strain.

9. The method of claim 1, wherein the cell is a Madin-Darby Canine Kidney (MDCK) cell, MA104 cells, Vero cell, EB66, or PER C6 cell.

10. The method of claim 1, further comprising incubating the infected cells under conditions suitable for the production of the virus by the cells and harvesting the virus.

11. A method of increasing Rotavirus production of one or more Rotaviruses comprising infecting a cell or cell line with a Rotavirus; incubating the infected cells under conditions suitable for the production of the virus by the cells, wherein the medium comprises an RNA polynucleotide that inhibits expression of a coding region selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51.

12. The method of claim 11, wherein the RNA polynucleotide is a siRNA, shRNA, miRNA mimic, miRNA inhibitor, or antisense polynucleotide.

13. A cell comprising reduced expression of at least one gene selected ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51.

14. The cell of claim 13, wherein the gene expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 is reduced at least 15% relative to a control.

15. The cell of claim 13, wherein the gene with reduced expression is NAT9.

16. The cell of claim 15, further comprising reduced expression of the ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 gene.

17. The cell of claim 13, comprising reduced expression of at least two genes selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPPSC, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51.

18. The cell of claim 13, wherein reduced expression of ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 occurs through a mutation in a regulator region operably linked to the coding region for ZNF205, NEU2, NAT9, SVOPL, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, COQ9, BTN2A1, PYCR1, EP300, SEC61G, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes.

19. The cell of claim 13, wherein reduced expression ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 occurs through direct targeting of the coding region of ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 with an exogenous control element.

20. The cell of claim 19, wherein the exogenous control element is a siRNA, shRNA, small molecule inhibitor, or antisense polynucleotide.

21. The cell of claim 13, wherein the cell is a Madin-Darby Canine Kidney (MDCK) cell, Vero cell, MA104 cells, EB66, or PER C6 cell.

22. A cell line comprising the cell of claim 13.

23. An engineered cell line comprising decreased expression of at least one gene selected from ZNF205, NEU2, NAT9, SVOPL, COQ9, BTN2A1, PYCR1, EP300, SEC61G, NDUFA9, RAD51AP1, COX20, MAPK6, WDR62, LRGUK, CDK6, KIAA1683, CRISP3, GRPR, DPH7, GEMIN8, KIAA1407, RFXAP, SMARRCA4, CCDC147, AACS, CDK9, C7ORF26, ZDHHC14, RNUT1, GAB1, EMC3, FAM96A, FAM36A, LOC55831, LOC136306, DEFB126, MGC955, EPHX2, SRGAP1, PPP5C, MET, SELM, TSPYL2, TSARG6, NDUFB2, PLAU, FLJ36888, ADORA2B, FLJ22875, HMMR, NRK, LRIT3, FLJ44691, GPR154, ZGPAT, DRD1, FLJ27505, EDG5, SNRNP40, HPRP8BP, GPA33, JDP2, FLJ20010, FOXJ1, SCT, CHD1L, SULT1C1, STN2, MRS2L, RAD51AP1, DPH7, CLPP, ZNF37, AP3B2, DEGS2, PIR, D2LIC, CNTF, PAM, MYH9, PRPF4, SLC4A11, LRRCC1, FZD9, GPR43, LTF, ARIH1, PIK3R3, PTGFRN, HSPA5BP1, ZDHHC16, KIAA1764, C19ORF14, FLNA, FLJ32786, DKFZP434K046, C9ORF112, and/or PIR51 genes relative to a control.
